SEQUENCE LISTING (10 sequences)

SEQ ID NO: 1 - DNA, 74 bp, Artificial Sequence
  DNA template for selection of aptamer
gagtgaccgtctgcctgnnnnnnnnnnnnnnnnnnnnnnnnnnnnnnnnnnnnnnnncagccacaccaccagcc

SEQ ID NO: 2 - DNA, 18 bp, Artificial Sequence
  5′ primer for amplification of aptamer
gcctgttgtgagcctcct

SEQ ID NO: 3 - DNA, 17 bp, Artificial Sequence
  3′ primer for amplification of aptamer
ggctggtggtgtggctg

SEQ ID NO: 4 - DNA, 17 bp, Artificial Sequence
  5′ primer for amplification of aptamer
gagtgaccgtctgcctg

SEQ ID NO: 5 - DNA, 17 bp, Artificial Sequence
  3′ primer for amplification of aptamer
ggctggtggtgtggctg

SEQ ID NO: 6 - DNA, 50 bp, Artificial Sequence
  EGFR aptamer, #1193-50
agttcagccccggnnanacggncncangccngngcgnnnaaccnagacca

SEQ ID NO: 7 - DNA, 40 bp, Artificial Sequence
  ERBB2 aptamer, #1194-34
nccnggcangnncganggaggccnnngannacagcccaga

SEQ ID NO: 8 - DNA, 42 bp, Artificial Sequence
  IR aptamer, #1652-36
gcctgnaaggnnnaagcnnggccnaanggngcnancaggcnc

SEQ ID NO: 9 - DNA, 49 bp, Artificial Sequence
  PSA aptamer, #1660-07
acgagnnngnggnnnnggacaggcagcngacgnngggcnncncgcaaca

SEQ ID NO: 10 - DNA, 50 bp, Artificial Sequence
  Akt2 aptamer, #2153-02-02
agttccnnnacccnnngnggncgacnnccnanggaggagnnnanggacca

(19) United States
(12) Patent Application Publication
(10) Pub. No.: US 2013/0059292 A1
(43) Pub. Date: Mar. 7, 2013

(54) METHOD OF DETECTING A TARGET USING APTAMER-MEDIATED PROTEIN PRECIPITATION ASSAY

(71) Applicants: Ki-Seok KIM, Pohang-si (KR); Seung-Jin Lee, Pohang-si (KR); Dong-Il Han, Seoul (KR); Sung-Ho Ryu, Pohang-si (KR); Sung-Key Jang, Pohang-si (KR)

(72) Inventors: Ki-Seok KIM, Pohang-si (KR); Seung-Jin Lee, Pohang-si (KR); Dong-Il Han, Seoul (KR); Sung-Ho Ryu, Pohang-si (KR); Sung-Key Jang, Pohang-si (KR)

(73) Assignee: Aptamer Sciences Inc., Pohang-si (KR)

(21) Appl. No.: 13/599,125

(22) Filed: Aug. 30, 2012

Related U.S. Application Data
(60) Provisional application No. 61/528,791, filed on Aug. 30, 2011.

(51) Int. Cl. G01N 33/53; C12Q 1/70; C12Q 1/68

(52) U.S. Cl. 435/5; 436/501; 435/6.1

(57) ABSTRACT

The present invention relates to a method and detection kit for determining a presence of one or more proteins using aptamer-mediated pull-down assays. Specifically, a reproducible a protein precipitation (Co-AP/AP) method is provided to identify physiologically relevant protein-protein interactions by using target protein-specific aptamers, and to confirm its superior performance over antibody based protein precipitation (Co-IP/IP) methods in terms of its protein pull-down performance.

### FIELD OF THE INVENTION

The present invention relates to a method and detection kit for determining a presence of one or more proteins using aptamer-mediated pull-down assays.

### BACKGROUND OF THE INVENTION

When a protein is expressed at low levels and is difficult to detect with western blot analysis, a protein pull down assay may be the method of choice. Protein pull down assay involves using an antibody that is specific for a known protein to isolate that particular protein from a solution containing many different proteins. These solutions will often be in the form of a crude lysate of a plant or animal tissue.

Protein pull down of intact protein complexes (i.e.: antigen along with any proteins or ligands that are bound to it) is known as co-immunoprecipitation (Co-IP). Co-IP is a popular technique to identify physiologically relevant protein-protein interactions by using target protein-specific antibodies. Co-IP works by selecting an antibody that targets a known protein that is believed to be a member of a larger complex of proteins. By targeting this known member with an antibody it may become possible to pull the entire protein complex out of solution and thereby identify unknown members of the complex.

The identification of interacting proteins of a particular target protein may help defining protein-protein interaction and proteins of unknown functions. To isolate protein complexes, high-speed ultracentrifugation, sucrose density-gradient centrifugation, co-immunoprecipitation (Co-IP), and epitope-tag affinity purification have been widely used. The most common approach to isolate a protein complex is by use of Co-IP. By use of a specific antibody raised against the target proteins, the interacting proteins can be co-purified. The immunoprecipitated protein complex can then be digested in the solution by the protease trypsin) followed by an MS analysis for protein identification. After MS analysis, protein identification is reached by the use of an algorithm against a sequence database.

The disadvantage of the Co-IP/IP is that the antibody may still have a chance to crossreact with other nonspecific proteins. A protein Co-IP/IP reagent has to be specific in order to avoid precipitation of unwanted proteins. Furthermore, sufficient affinity is required to pull down the protein and it has to withstand stringent washing steps. A specific antibody that can recognize and specifically bind to the protein of interest has been widely used for this purpose. However, the production of a specific antibody is time-consuming and not a foolproof procedure. And many antibodies are unfaithful to their cognate target proteins due to the inefficient detection of endogenous target proteins in Co-IP/IP assay. The most commonly encountered problems with the IP and Co-IP approach is the concomitant recovery of large amounts of antibody. Co-elution of antibody fragments with antigen often results in bands interfering with detection of any co-precipitated proteins on SDS-PAGE and mass analysis. Accordingly, there is a need for an alternative method to identify physiologically relevant protein-protein interactions.

Aptamers are single-stranded oligonucleotides that form stable three-dimensional structures capable of binding with high affinity and specificity to a variety of molecular targets. Aptamers bind to protein targets in much the same manner as antibodies and modulate protein function. Aptamers against a specific target are generated using an iterative approach called SELEX (Systematic Evolution of Ligands by Exponential Enrichment) (Science, 1990, 249, 505-510). It is possible to produce an aptamer with a high affinity for a small molecule, such as a peptide or other molecular compound, against which antibodies are difficult to obtain. Aptamers have advantages over more antibodies in that they are poorly immunogenic, stable, and often bind to a target molecule more strongly than do antibodies. Aptamers can only bind to target proteins with native conformational structure, against which antibodies are difficult to bind. Furthermore, producing an aptamer is more cost-advantageous than an antibody because it can be synthesized easily and in large quantities by in vitro transcription, PCR, or chemical synthesis (Annu. Rev. Med. 2005, 56, 555-583; Nat. Rev. Drug Discov. 2006, 5, 123-132). Thus, aptamers are useful and cost-effective tools for biochemical analyses. Also, they can be generated quickly against multiple targets. Aptamers have been shown to be useful as therapeutic agents (J. Biotechnol., (2000, 74, 5-13), diagnostic tools (J. Biotechnol., (2000, 74, 5-13), biochemical detection (Anal. Biochem., 2008, 375, 217-222), and affinity-purification (Nat. Rev. Microbiol., 2006, 4, 588-596; RNA, 2008, 14, 1154-1163). The aptamer based protein precipitation (Co-AP and AP) which is a technique to identify physiologically relevant protein-protein interactions by using target protein-specific aptamers and aptoprecipitation assay with reduced nonspecific binding of protein to beads is provided. The aptamer based protein precipitation methods show superior performance compared to antibody based protein precipitation (Co-IP and IP) methods for subsequent protein identification by Western blotting or mass spectrometry from solution digests.

### SUMMARY OF THE INVENTION

Embodiments are directed to a method and detection kit for determining a presence of one or more target proteins using aptamer-mediated protein precipitation assay.

The embodiments may be realized by providing a method of determining a presence of a protein in a fluid sample, said method comprising the steps of:
a) providing a solid substrate bound aptamer, wherein the aptamer is a single-stranded nucleic acid having 20 to 200 nucleotides capable of specifically binding to a target protein;
b) contacting the fluid sample with the solid substrate bound aptamer to form a solid substrate bound aptamer-target protein complex when the target protein is present in the fluid sample; and
c) determining whether the protein is present in the fluid sample by isolating and identifying the target protein from the solid substrate bound aptamer-target protein complex or detecting the formation of the solid substrate bound aptamer-target protein complex, wherein the protein to be determined is the target protein of the aptamer used.

The protein may further comprise an interacting protein of the target protein.

The fluid sample may be at least one selected from serum, spinal fluid, cerebrospinal fluid, joint fluid or one produced by contacting a lysis buffer with at least one selected from mammalian cells, yeasts, virus, or prokaryotic cells.

The method may further comprise a step introduced prior to, after, or simultaneously with step b) of contacting the fluid sample or the solid substrate bound aptamer-target protein complex with at least one selected from an oligonucleotide, or a polymer with charge, to remove undesired proteins.

The polymer with charge may be at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

The concentration of the polymer with charge in the fluid sample may range from 0.01 to 10 mM.

The method may further comprise a step introduced prior to, after, or simultaneously with step b) of contacting the fluid sample or the solid substrate bound aptamer-target protein complex with at least one selected from an oligonucleotide, or a polymer with charge, to remove undesired proteins.

The polymer with charge may be at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

The concentration of the polymer with charge in the fluid sample may range from 0 to 0.1 mM.

The embodiments may also be realized by providing a detection kit for determining a presence of a protein in a fluid sample, said kit comprises:

a solid substrate;

an aptamer bound to said solid substrate, wherein the aptamer is a single-stranded nucleic acid having 20 to 200 nucleotides capable of specifically binding to a target proteins; and

a detection means for identifying the formation of a solid substrate bound aptamer-target protein complex,

wherein the protein to be detected by the detection means is the target protein of the aptamer used.

The protein may further comprise an interacting protein of the target protein.

The kit may further comprise a polymer with charge, an oligonuceleotide, or a combination thereof.

The polymer with charge may be at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

The concentration of the polymer with charge in the fluid sample may range from 0.01 to 10 mM.

The kit may further comprise a polymer with charge, an oligonuceleotide, or a combination thereof.

The polymer with charge may be at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

The concentration of the polymer with charge in the fluid sample may range from 0 to 0.1 mM.

The aptamer may be one or more selected from the group consisting of SEQ ID NOs 6 to 10.

An oligonucleotide molecule having the nucleotide sequence selected from the group consisting of SEQ ID NOs 6 to 10 may be provided.

A reproducible a protein precipitation (Co-AP/AP) method is provided to identify physiologically relevant protein-protein interactions by using target protein-specific aptamers, and to confirm its superior performance over antibody based protein precipitation (Co-IP/IP) methods in terms of its protein pull-down performance and also for subsequent protein identification by Western blotting or mass spectrometry from solution digests. In addition to its superior pull down performance, Co-AP/AP method may show some additional benefits. Mild elution conditions gives non-denatured proteins which can be used for further functional study of endogenous proteins and no co-elution of any interfering bands which limits subsequent analysis using mass spectrometry.

The introduction of oligonucleotides and polymer with charge may be used for enhancing its overall protein pull down performance and also controlling pull down specificity. Aptamers would be applicable as a useful and cost-effective tool for identification of a particular target protein with its interacting proteins.

### DETAILED DESCRIPTION OF THE INVENTION

Molecules that recognize others with extreme specificity and high-affinity are important for a wide range of applications. Typically, antibodies fulfill this role in immunoassays. Recent advancement of research has led to the discovery of a class of oligonucleotides referred to as aptamers that can recognize molecules with high-affinity and specificity. Consequently, aptamers have the potential to fulfill the role that antibodies play in research applications.

Generally, aptamers are oligonucleotides selected from random-sequence, single-stranded nucleic acid libraries by an in vitro selection and amplification procedure known as SELEX (systematic evolution of ligands by exponential enrichment). The selected aptamers are small single-stranded nucleic acids that fold into a well defined three-dimensional structure. They show a high affinity and specificity for their target molecules and inhibit their biological functions.

An emerging direction in the aptamer field is the systematic identification of target molecules that are associated with distinct cellular states. The identification of new therapeutically and diagnostically relevant biomolecules can be facilitated with the goal of developing an individualized medical approach. In this regard, aptamers, the cognate target molecules on the cell surfaces can be subsequently identified by using aptamer-based protein precipitation (Co-AP and AP) protocols followed by SDS-PAGE, protease digestion, and liquid chromatography/mass spectrometry (LC-MS) analysis.

Although aptamers have been tested in various diagnostic platforms, a Co-AP and AP assay that is completely based on aptamers has not yet been reported. Specifically, aptoprecipitation (AP) refers to a technique of precipitating a protein from a solution using an aptamer that specifically binds to that particular protein. This process can be used to isolate and concentrate a particular protein from a sample containing many thousands of different proteins. Co-aptoprecipitation of intact protein complex (i.e. target protein along with any proteins or ligands that are bound thereto) is known as co-aptoprecipitation (Co-AP). Co-AP works by selecting an aptamer that targets a known protein which belongs to a member of a larger complex of proteins. By targeting this known member with an aptamer it may become possible to pull the entire protein complex out of a solution, and thereby identify unknown members of the complex.

The present invention relates to a protein precipitation assay using aptamers and the detection of a specific target molecule and identification of interacting proteins of a particular target protein in cells while significantly reducing the nonspecific proteins.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. In addition, it will also be understood that when a layer is referred to as being “between” two layers, it can be the only layer between the two layers, or one or more intervening layers may also be present. Like reference numerals refer to like elements throughout.

The terms “nucleic acid” “polynucleotide” and “oligonucleotide” are used interchangeable herein and refer to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Aptamers are oligonucleic acid molecules that bind to a specific target molecule. Typically, aptamers are DNA or RNA oligonucleotides selected from random-sequence, single-stranded nucleic acid libraries by an in vitro selection and amplification procedure known as SELEX. For examples of SELEX processes see U.S. Pat. Nos. 5,270,163; 5,475,096; and 5,567,588, which are incorporated herein by reference in their entirety.

The present invention provides methods of determining a presence of one or more targets of interests in a fluid sample using an aptamer based assay.

An embodiment of the invention provides a method of determining a presence of a protein in a fluid sample, said method comprising the steps of:
a) providing a solid substrate bound aptamer, wherein the aptamer is a single-stranded nucleic acid having 20 to 200 nucleotides capable of specifically binding to a target protein;
b) contacting the fluid sample with the solid substrate bound aptamer to form a solid substrate bound aptamer-target protein complex when the target protein is present in the fluid sample; and
c) determining whether the protein is present in the fluid sample by isolating and identifying the target protein from the solid substrate bound aptamer-target protein complex or detecting the formation of the solid substrate bound aptamer-target protein complex, wherein the protein to be determined is the target protein of the aptamer used.

The target proteins may include, but are not limited to, growth factors, growth factor receptors (e.g., epidermal growth factor receptor (EGFR), Human Epidermal growth factor Receptor 2 (ErbB2), and the like), insulin receptor (IR), Prostate-specific antigen (PSA), serine/threonine protein kinase (e.g., Akt protein such as Akt 1 and Akt 2, and the like), and the like.

The aptamer is a single-stranded nucleic acid having 20 to 200 nucleotides, and preferably 30 to 100 nucleotides, and has a high affinity and specificity to a target protein of interest, thereby specifically binding to the target protein of interest. The aptamer that is capable of specifically binding to the target protein of interest may be selected from random-sequence, single-stranded nucleic acid libraries by an in vitro selection and amplification procedure known as SELEX. The selected aptamers are small single-stranded nucleic acids that fold into a well defined three-dimensional structure. They show a high affinity and specificity for their target molecules and inhibit their biological functions.

Aptamer may comprise modified base for increasing the affinity and specificity for their target protein. For example, the modified base may be a pyrimidine modified by a hydrophobic group, such as benzyl group, a naphthyl group, or a pyrrolebenzyl group, at its 5- position. Modified nucleoside may be exemplified as 5-(N-benzylcarboxyamide)-2′-deoxyuridine (called BzdU), 5-(N-naphthylcarboxyamide)-2′-deoxyuridine (called NapdU), 5-(N-4-pyrrolebenzyl carboxyamide)-2′-deoxyuridine (called 4-PBdU), 5-(N-benzylcarboxyamide)-2′-deoxycytidine (called BzdC), 5-(N-naphthylcarboxyamide)-2′-deoxycytidine (called NapdC), 5-(N-4-pyrrolebenzylcarboxyamide)-2′-deoxycytidine (called 4-PBdC), 5-(N-benzylcarboxyamide)-2′-uridine (called BzU), 5-(N-naphthylcarboxyamide)-2′-uridine (called NapU), 5-(N-4-pyrrolebenzylcarboxyamide)-2′-uridine (called 4-PBU), 5-(N-benzylcarboxyamide)-2′-cytidine (called BzC), 5-(N-naphthylcarboxyamide)-2′-cytidine (called NapC), 5-(N-4-pyrrolebenzyl carboxyamide)-2′-cytidine (called 4-PBC), and the like, but not be limited thereto.

For example, the aptamer may be selected from the group consisting of the group SEQ ID NO: 6, SEQ ID NO: 7, SEQ ID NO: 8, SEQ ID NO: 9, and SEQ ID NO: 10, but not be limited thereto.

The protein may further comprise an interacting protein of the target protein. In other words, in one embodiment of the instant invention, a Co-AP assay method based on aptamers which detects the interacting proteins of a particular target protein in cell lysate is provided.

The fluid sample may be derived from contacting a cell and/or a tissue with a lysis buffer. More specifically, the fluid sample may be at least one selected from serum, spinal fluid, cerebrospinal fluid, joint fluid or one produced by contacting a lysis buffer with at least one selected from mammalian cells, yeasts, virus, or prokaryotic cells.

The method may further comprise a step introduced prior to, after, or simultaneously with step b) of contacting the fluid sample or the solid substrate bound aptamer-target protein complex with at least one selected from an oligonucleotide, or a polymer with charge, to remove undesired proteins.

The oligonucleotide may be a DNA and/or RNA from a prokaryotic cell, a eukaryotic cell, or a mixture thereof; for example, the oligonucleotide may be a DNA and/or RNA of salmon sperm, calf thymus, Herring, or a mixture thereof.

The oligonucleotide may reduce non-specific bindings when reacting with a cell lysate during the pull-down process. Specifically, the non-specific binding may be caused by the presence of intracellular nuclease as well transcription factors. For example, a salmon sperm may serve a role as a blocking agent to minimize the nuclease or transcription factors interfering with aptamers.

The concentration of the oligonucleotide in the fluid sample may range from 50 ug/mL to 200 ug/mL.

The polymer with charge may be at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

The average molecular weight of the polymer with charge may be greater than 1,000,000 Da.

The concentration of the polymer with charge may range from 0 to 10 mM. Depending on the concentration used with the range, either AP or Co-AP may be selectively performed. In particular, to effectively achieve the effect of reducing non-specific bindings in AP, the concentration of the polymer with charge in the fluid sample may range from 0.01 to 10 mM, preferably from 0.1 to 10 mM, and more preferably from 1 to 10 mM.

In Co-AP, to effectively achieve the effect to identify physiologically relevant protein-protein interactions as well as reducing non-specific bindings, the concentration of the polymer with charge in the fluid sample may range from 0 to 0.1 mM, and preferably from 0 to 0.05 mM.

The solid substrate may be magnetic or non-magnetic beads selected from the group consisting of sepharose beads, agarose beads, Dyna beads, agarose magnetic beads, sepharose magnetic beads, streptavidin Dyna beads, streptavidin agarose beads, and streptavidin sepharose beads, but not limited thereto.

The step of isolating the one or more target proteins from the solid substrate bound aptamer-target protein complex or the solid substrate bound receptor-ligand-aptamer-target protein complex may be performed by any conventional method known to the relevant field, for example, DNase, non-conjugated aptamer, EDTA, acid, alkaline, or a combination thereof, but not limited thereto.

The step of detecting whether the target protein, or the solid substrate bound aptamer-target protein complex or the solid substrate bound receptor-ligand-aptamer-target protein complex is present in the reaction mixture may be performed by any conventional method known to the relevant field, for example, fluorescence detection, mass spectrometry, chromatography, electrophoresis, or a combination thereof, but not limited thereto.

Also provided herein are methods for substantially reducing background noise of nonspecific proteins encountered in AP assay. Some aspects of the methods eliminate or significantly reduce the phenomenon of nonspecific protein. Such reduction or elimination of nonspecific interaction is useful in a wide variety of proteomics applications.

Co-AP assay and AP assays based on aptamers resulted in a significantly less interference and higher specificity than those using antibodies. Co-AP or AP assay based on aptamers can be used in a variety of applications including, but not limited to, detection of marker proteins in serum samples and target molecules in cells. It can also be used for identification of novel proteins, development of new diagnostic tools, and development of novel therapeutic tools.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### BRIEF DESCRIPTION OF THE DRAWINGS

FIGS. 1A and 1B show the results of SYPRO Ruby stain and Western blot, respectively, of AP assay using aptamers.

FIGS. 2A and 2B show the results of SYPRO Ruby stain and Western blot, respectively, of improvement of AP (pull-down) performance using salmon sperm DNA (ssDNA).

FIG. 3 shows the results of SYPRO Ruby stain showing reduction of nonspecific proteins by using dextran sulfate (DxSO₄) in AP assay

FIG. 4 shows the results of SYPRO Ruby stain showing reduction of nonspecific proteins by using polyanions in AP assay.

FIGS. 5A, 5B, 5C, 5D show the results of SYPRO Ruby stain of determination of elution condition in Co-AP assay

FIG. 6 show the results of Western blot of Co-AP assay using insulin receptor aptamers.

FIG. 7 show the results of Western blot of Co-AP assay using AKT2 aptamers.

FIG. 8 show the results of SYPRO Ruby stain of Co-AP assay using EGFR aptamers.

FIG. 9 show the results of Western blot of Co-AP assay using EGFR aptamers.

FIG. 10 show the results of SYPRO Ruby stain of Co-AP and Co-IP assay using EGFR aptamers and antibodies, respectively.

FIG. 11 show the results of Western blot of Co-AP and Co-IP assay using EGFR aptamers and antibodies, respectively.

FIG. 12 show the results of Venn diagram showing the overlap of nonspecific interaction proteins and Akt interacting parteners in MCF7 cells.

FIG. 13A show the results of Venn diagram showing the overlap of nonspecific interaction proteins and EGFR interacting parteners using EGFR aptamer in MCF7 cells.

FIG. 13B show the results of Venn diagram showing the overlap of nonspecific interaction proteins and EGFR interacting parteners using EGFR antibody in MCF7 cells.

FIG. 14 shows the results of SYPRO Ruby stain showing comparison of performance of aptamer and antibody in pull-down assay.

FIG. 15 and FIG. 16 show another result of SYPRO Ruby stain showing comparison of performance of aptamer and antibody in pull down assay.

FIGS. 17A, 17B, 17C, and 17D show the results of SYPRO Ruby stain showing AP performance of aptamer in 10% serum.

FIGS. 18A, 18B, 18C, and 18D show the results of SYPRO Ruby stain showing AP performance of aptamer in HEK293 cell lysates.

FIGS. 19A and 19B show the results of SYPRO Ruby stain showing AP performance of conjugated aptamer (amine aptamer-sepharose magnetic beads) in HEK293 cell lysates.

FIGS. 20A, 20B, and 20C show the results of SYPRO Ruby stain showing AP performance of conjugated aptamer (thiol aptamer-magnetic agarose beads) in HEK293 cell lysates.

FIGS. 21A, 21B, and 21C show the results of SYPRO Ruby stain showing AP performance of conjugated aptamer (thiol aptamer-magnetic agarose beads) in various cell lysates.

FIG. 22 shows the results of SYPRO Ruby stain and MALDI-MS/MS showing comparison of performance of aptamer and antibody in AP assay.

FIGS. 23A and 23B show another result of SYPRO Ruby stain and western blot showing comparison of performance of aptamer and antibody in AP assay.

FIGS. 24A and 24B show still another result of SYPRO Ruby stain and western blot showing comparison of performance of aptamer and antibody in AP assay.

### EXAMPLES

### Example 1

### Material and Methods

### 1.1: SELEX (Systematic Evolution of Ligands by Exponential Enrichment)

Synthetic DNA template containing 40 random nucleotides flanked by fixed regions 5′-GAGTGACCGTCTGCCTG-40N-CAGCCACACCACCAGCC-3′ (SEQ ID NO: 1) complementary to the primers 5′-BB-GCCTGTTGTGAGCCTCCT-3′ (SEQ ID NO: 2) and 5′-GGCTGGTGGTGTGGCTG-3 (SEQ ID NO: 3), where BB denotes three biotin phosphoramidite couplings, were amplified by PCR (Perkin-Elmer/Cetus thermal cycler). Twenty-five thermal cycles were conducted at 93° C. for 30 sec, 52° C. for 20 sec, and 72° C. for 60 sec. The SELEX process was performed at 37° C. A mixture of 1 mmol of aptamer library dissolved in a buffer solution (40 mM HEPES/pH 7.5, 120 mM NaCl, 5 mM KCl, 5 mM MgCl₂, 0.002% tween-20) was heated at 95° C. for about 3 min, and then slowly cooled to 37° C. at 0.1° C./sec for aptamer re-folding. Solutions of 0.1% BSA, 10 mM Prothrombin, and 10 mM casein were added to the buffer to eliminate non-specific aptamer. Aptamer library was pre-incubated with His tag magnetic bead (Invitrogen) to eliminate non-specific binder to magnetic bead. Aptamer library in supernatant were incubated with purified 10 pmol of target proteins for 30 min and then target proteins captured by contacting with His tagged beads for 10 min. The captured target proteins were separated in magnetic field and then washed three times with the buffer. Aptamers bound to the target proteins were eluted with 2 mM NaOH solution and amplified via PCR reaction with biotinylated 5′-primer. The resulting aptamers were used in the next SELEX round.

### 1.2: Cloning and Sequencing

Aptamer pools were amplified by 5′-primer (GAGTGACCGTCTGCCTG, SEQ ID NO: 4) and 3′-primer (GGCTGGTGGTGTGGCTG, SEQ ID NO: 5), and cloned into pUC9 plasmid (Solgent). Sixty individual plasmids were sequenced using DYEnamic ET-terminator cycle sequencing premix kit (Amersham Pharmacia Biotech, Inc., Piscataway, N.J.) and ABI Prism 377 sequencer.

### 1.3: Oligonucleotide Synthesis, Purification, and Characterization

All oligonucleotides were synthesized on functionalized controlled pore glass (CPG) (BioAutomation) on an automated solid phase DNA synthesizer (Mermade12, BioAutomation) using 0.067 M solution of the 5-(N-benzylcarboxyamide)-2′-deoxyuridine-amidite or the 5-(N-napyl lcarboxyamide)-2′-deoxyuridine-amidite in anhydrous acetonitrile. For incorporation of dA, dG, dC and dT residues standard phosphoramidites (Proligo) with exocyclic amino groups protected with benzoyl group (for dA and dC) and isobutyryl group (for G) were used. For incorporation of 5-(N-benzylcarboxyamide)-2′-deoxyuridine-amidite, phosphoramidite solution was delivered in two portions, each followed by a 5 min coupling wait time. Oxidation of the internucleotide phosphate to phosphate was carried out using an oxidizer [tetrahydrofuran (THF), pyridine, 0.02 M iodine and water] with waiting time. All other steps in the protocol supplied by the manufacturer were used without modification. After completion of the synthesis, the resulting mixture was treated with a cleavage solution (t-butylamine:methanol:water, 1:1:2, v/v) at 70° C. for 5 hours to hydrolyze the ester linking the DNA to the support and to remove protecting groups from the purine and pyrimidine bases. The resulting mixture was frozen, filtered, and speed-vac evaporated to dryness.

Crude oligonucleotides were purified by high performance liquid chromatography (AKTA basic HPLC, XBridge OST C18 10×50 mm, A=100 mM buffer triethylammoniumbiocarbonate (TEAB), pH=7, B=acetonitrile, 8% to 40% B in 20 min, flow rate of 5 mL/min, at 65° C., l=254 and 290 nm). Oligonucleotides were characterized by LC ESI-MS and purities were assessed by HPLC.

### 1.4: Binding Affinity Assays

The aptamer-protein equilibrium dissociation constants (Kd's) were determined by the nitrocellulose-filter binding method (Mol Ther. 2001, 4, 567-573). For all binding assays, aptamers were dephosphorylated using alkaline phosphatase (New England Biolab) and 5-end labeled using T4 polynucleotide kinase (New England Biolabs, Beverly, Mass.) and [³²P]-ATP (Amersham Pharmacia Biotech, Piscataway, N.J.) (Methods Enzymol. 1996, 267, 275-301). Before binding assay, aptamer was heated at 95° C. for 3 min, and then slowly cooled to 37° C. at 0.1° C./sec in buffer (40 mM HEPES/pH 7.5, 120 mM NaCl, 5 mM KCl, 5 mM MgCl₂, 0.002% tween-20) to allow for aptamer refolding. Direct binding assays were carried out by incubating ³²P-labeled aptamer at a concentration of less than 10 μM and protein at concentrations ranging from 1 mM to 10 fM in selection buffer at 37° C. The fraction of bound aptamer was quantified with a PhosphorImager (Fuji FLA-5100 Image Analyzer, Tokyo, Japan). Raw binding data were corrected for nonspecific background binding of radiolabeled aptamer to the nitrocellulose filter and reported as the mean±standard error of the mean (SEM) of three experiments.

Target-specific aptamers were designed and synthesized (Table 1). All of aptamers incorporated with 5-BzdU or 5-NapdU-modified oligonucleotides were prepared for the Co-AP or AP assay. The aptamers shown in Table 1 were obtained by minimizing the length of amino acids of SEQ ID No. 1 while maintaining the aptamer-protein equilibrium dissociation constants (Kd's).

TABLE 1
A list of sequence of aptamers used for Co-AP and AP assay

| Aptamers | Sequence (5′→3′) |
|---|---|
| EGFR (#1193-50) | AGTTC AGCCCCGG66A6ACGG6C6CA6GCC6G6GCG666AACC6AGACCA (SEQ ID NO: 6) |
| ERBB2 (#1194-34) | 6CC6GGCA6G66CGA6GGAGGCC666GA66ACAGCCCAGA (SEQ ID NO: 7) |
| IR (#1652-36) | GCCTG5AAGG555AAGC55GGCC5AA5GG5GC5A5CAGGC5C (SEQ ID NO: 8) |
| PSA (#1660-07) | ACGAG555G5GG5555GGACAGGCAGC5GACG55GGGC55C5CGCAACA (SEQ ID NO: 9) |
| Akt (#2153-02-02) | AGTTCC555ACCC555G5GG5CGAC55CC5A5GGAGGAG555A5GGACCA (SEQ ID NO: 10) |

In Table 1, ‘5’ indicates the 5-BzdU-modified oligonucleotide and ‘6’ indicates the 5-NapdU-modified oligonucleotide of aptamers.

### Example 2

### Cell Culture

The human embryonic kidney cell line (HEK293) (POSTECH, Signal transduction laboratory, Sung Ho Ryu) the human skin carcinoma cell line (A431) (POSTECH, Signal transduction laboratory, Sung Ho Ryu) and Rat-1 cells stably expressing the human insulin receptor (Rat-1/IR) (POSTECH, Signal transduction laboratory, Sung Ho Ryu) were cultured in Dulbecco's modified Eagle's medium (DMEM) (Invitrogen, Grand Island, N.Y.) supplemented with 10% fetal bovine serum (Gibco, Grand Island, N.Y.), 10 U/mL penicillin and 10 μg/mL streptomycin (Gibco, Grand Island, N.Y.) in a 5% CO₂-humidified chamber at 37° C. The human breast adenocarcinoma cell line (MCF7) (POSTECH, Signal transduction laboratory, Sung Ho Ryu) and MCF7 cells stably expressing the human ERBB2 (MCF7/ERBB2) (POSTECH, Signal transduction laboratory, Sung Ho Ryu) were cultured in RPMI-1640 medium (Invitrogen, Grand Island, N.Y.) supplemented with 10% fetal bovine serum (Gibco, Grand Island, N.Y.), 10 U/mL penicillin and 10 μg/mL streptomycin (Gibco, Grand Island, N.Y.) in a 5% CO₂-humidified chamber at 37° C.

### Example 3

### Conjugation of Aptamer to Beads

Aptamers were immobilized to NHS-activated Sepharose beads according to the manufacturer's instructions. Briefly, NHS-activated Sepharose or NHS-activated Sepharose magnetic 4 Fast Flow beads (GE Healthcare) were prepared with 1 mM HCl three times for pre-activation. NH₂-aptamer diluted in coupling buffer (0.2 M NaHCO₃, 0.5 M NaCl, pH 8.3) was added to the NHS beads. Beads were washed with coupling buffer (0.2 M NaHCO₃, 0.5 M NaCl, pH 8.3) followed by shaking for 2 hrs at ambient temperature on a rocker. Quenching was performed with quenching buffer (0.5 M NaCl, 0.1 M Tris-HCl, pH 8.5). Beads were washed with PBS three times.

SH-aptamers were immobilized to Magnetic beads-NH2 (Dynalbeads M270 amine) (143-(07D, Invitrogen) or amino magnetic agarose beads (BioScience Beads Division). Pack 0.5 ml magnetic beads in column or centrifuge tube and wash thoroughly with activation buffer (1M NaCl, 0.05M NaHPO4, 1 mM EDTA pH 7.4). Following wash, the beads were prepared 50% suspension in the column with the activation buffer and added iodoacetic NHS (FW 283; Sigma/Aldrich #I9760) to 15 umoles/ml packed gel. Beads were incubated with shaking for 2 hours at ambient temperature. Acetic anhydride was added in a final concentration of 0.05M in the 50% suspension. Beads were washed with coupling buffer (N2 bubbled) (1M NaCl, 0.05M-Bicarb-NaOH, 1 mM EDTA pH 9.0). The activated thiol aptamer was added directly to the activated magnetic beads under N₂ and allowed to couple overnight. Supernatants were removed to check coupling efficiency. It was resuspend to about 50% suspension in the pH 9.0 buffer and add 2-mercaptoethanol to final 0.1M at least 2 hours with mixing at ambient or overnight blocking.

### Example 4

### Aptamer Based Co-Precipitation (Co-Aptoprecipitation, Co-AP) Assay

When cells were subconfluent (80˜90%), they were starved to serum-free medium overnight (16-18 h) and were stimulated with 100 nM insulin or EGF (Sigma) for 5 min at 37° C. Cell monolayers were washed twice with phosphate buffered saline (PBS) and lysed into lysis buffer (50 mM Tris, pH 7.5, 150 mM NaCl, 5 mM MgCl2, 1 mM EDTA, 5% glycerol, 1% NP-40) containing protease inhibitor cocktail (Roche) and phosphatase inhibitor cocktail (Roche) by incubation for 30 min on ice. The cell lysates were clarified by centrifugation at 12,000×g for 10 min at 4°. The cleared lysates were mixed with aptamer coupled to magnetic agarose bead, 200 μg/ml salmon sperm DNA (ssDNA) (Ambion), and final 0.01 mM dextran sulfate (DxSO₄). After incubating overnight at 4° C., the mixed solution washed four times with detergent free lysis buffer. Bound proteins were eluted with DNase I (Epicentre) or 20 mM EDTA in 40 mM HEPES buffer containing 100 mM NaCl. The bounded proteins or eluate were subjected to SDS-PAGE and stained by SYPRO ruby proteins (Invitrogen). Stained images were visualized using a Fuji FLA-5100 Image Analyzer. For identifying protein complex, the eluate can be digested by sequencing grade trypsin (Promega). The aptoprecipitated protein complex can then be subjected to an MS analysis for protein identification.

### Example 5

### Aptamer Based Precipitation (Aptoprecipitation, AP) Assay

About 4×10⁶ cells (70-80% confluence) in 100 mm culture dish were solubilized in lysis buffer (50 mM Tris, pH 7.5, 150 mM NaCl, 5 mM MgCl2, 1 mM EDTA, 5% glycerol, 1% NP-40) containing protease inhibitor cocktail, and the resulting mixture was incubated on ice for 10 min. The cell lysates were clarified by centrifugation at 12,000×g for 10 min at 4° after brief sonication. The cleared lysates were mixed with either biotinylated aptamer or magnetic agarose beads coupled aptamer, 200 μg/ml salmon sperm DNA (ssDNA) (Ambion), and final 1 mM dextran sulfate. After incubating for about 2 hr or overnight at 4° C., the mixed solution washed four times with detergent free wash buffer. Bound proteins were eluted with DNase I (Epicentre) or 40 mM HEPES (pH 8.0) buffer containing 20 mM EDTA and 100 mM NaCl. Total cell lysates or eluate were subjected to SDS-PAGE and stained by SYPRO ruby proteins (Invitrogen). Stained images were visualized using a Fuji FLA-5100 Image Analyzer.

For Western Blot Analysis, bound proteins were eluted by boiling them in a loading buffer. Total cell lysates or eluate were subjected to SDS-PAGE and blotted onto nitrocellulose membranes. After blocking in 5% skim milk in TBS-T (50 mM Tris-HCl, pH 7.5, 150 mM NaCl, 0.05% Tween 20), membranes were probed with specific antibodies and proteins were visualized with peroxidase-coupled secondary antibodies using the ECL system (Amersham Biosciences).

### Example 6

### Improvement of Aptoprecipitation (AP) Performance by Using Salmon Sperm DNA

AP assay with biotinylated DNA aptamers was performed to validate the specificity and affinity of aptamers towards EGFR (epidermal growth factor receptor). A431 cell lysates corresponding to 100 μg of total cellular proteins that express the EGFR were prepared and incubated with the 20 pmol biotinylated EGFR aptamers (#1193-50) followed by incubation with 10 μl strepavidin magnetic beads (MyOne™, Invitrogen). After washing the beads, the bound complex was eluted by boiling SDS loading buffer and SDS-PAGE gel (4-15% gradient gel) was stained with SYPRO ruby (Invitrogen). Western blot analysis was by the anti-EGFR antibodies (Cell signaling) after AP with the biotinylated EGFR aptamers in A431 cell lysates.

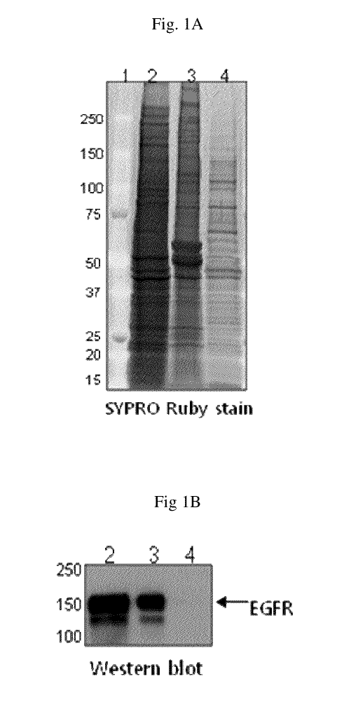

In FIGS. 1A and 1B, Lane 1 indicates protein marker; Lane 2 indicates A431 cell lysates corresponding to 20 μg of total cellular proteins; Lane 3 indicates supernatant after AP with the biotinylated EGFR aptamers; and Lane 4 indicates eluate by boiling SDS loading buffer from the beads bound complex.

Protein bands on gel were directly detected by SYPRO ruby proteins and Western blotting. No significant EGFR bands were detected on either gel (FIG. 1A) or in autoradiogram (FIG. 1B), indicating that the aptamers can be preferentially bound to DNA binding proteins such as transcription factors in the nucleus or aptamers can be degraded by nuclease in cell lysates.

In order to solve this problem in AP assays using aptamers, AP experiments were performed using salmon sperm DNA (ssDNA) as a competitor against DNA binding proteins or/and nuclease in cells. Different concentrations of ssDNA ranging from 50 to 200 μg/mL were used to block the access of aptamers to DNA binding proteins such as transcription factors or/and to inhibit nuclease attack in cells. A431 cell lysates corresponding to 100 μg of total cellular proteins that express the EGFR were prepared and incubated with the 20 pmol biotinylated EGFR aptamers (#1193-50) and 200 μg/mL ssDNA followed by incubation with 10 μl strepavidin magnetic beads (MyOne™, Invitrogen). After washing the beads, the bound complex was eluted by boiling SDS loading buffer and SDS-PAGE gel (4-15% gradient gel) was stained with SYPRO ruby (Invitrogen). Western blot analysis was by the anti-EGFR antibodies (Cell signaling) after AP with the biotinylated EGFR aptamers in A431 cell lysates.

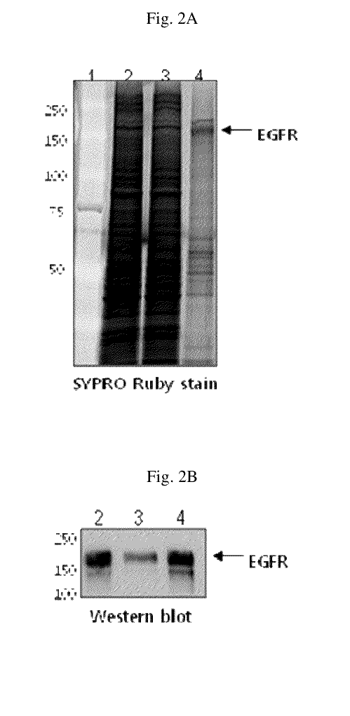

In FIGS. 2A and 2B, Lane 1 indicates protein marker; Lane 2 indicates A431 cell lysates corresponding to 20 μg of total cellular proteins; Lane 3 indicates supernatant after AP with the biotinylated EGFR aptamers; and Lane 4 indicates eluate by boiling SDS loading buffer from the beads bound complex.

A significant amount of EGFR bands were captured on both gel (FIG. 2A) and in autoradiogram (FIG. 2B). These data indicate that ssDNA block the access of aptamers to DNA binding proteins such as transcription factors or/and protects aptamer from nuclease attack in cells. Therefore, addition of ssDNA allowed aptamer to interact with the EGFR in cell lysates.

### Example 7

### Reduction of Nonspecific Proteins by Using Polyanions in AP Assay

To reduce background nonspecific proteins encountered in AP, the AP experiment was conducted using dextran sulfate (DxSO₄) as a competitor. Different concentrations of dextran sulfate ranging from 0.01 to 1.0 mM were used to block nonspecific proteins in cell lysates. A431 cell lysates corresponding to 100 μg of total cellular proteins that express the EGFR were prepared and incubated with the 20 pmol biotinylated EGFR aptamers (#1193-50) and Dextran sulfate followed by incubation with 10 μl strepavidin magnetic beads (MyOne™, Invitrogen). After washing the beads, the bound complex was eluted by boiling SDS loading buffer. The eluate was loaded on a SDS-PAGE (4-15% gradient gel). Protein bands on gel were directly detected by SYPRO ruby.

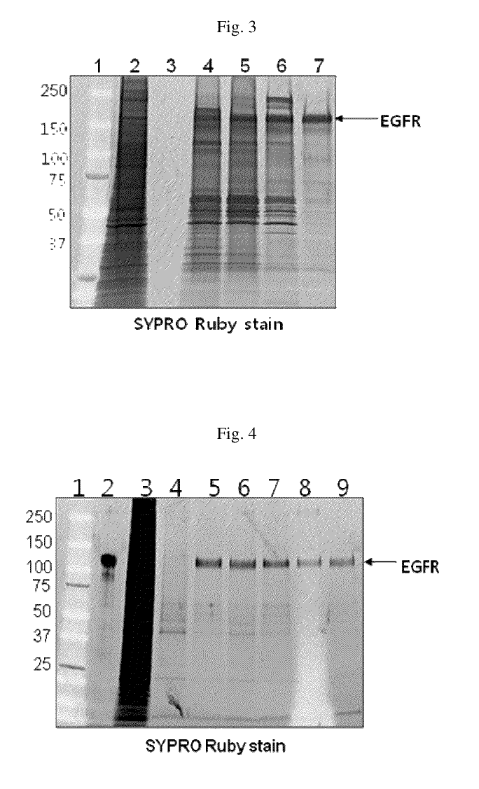

The results are shown in FIG. 3, wherein Lane 1 indicates protein marker; Lane 2 indicates A431 cell lysates corresponding to 20 μg of total cellular proteins; Lane 3 indicates blank; Lane 4 indicates eluate by boiling SDS loading buffer from the beads bound complex after AP with the biotinylated EGFR aptamers in A431 cell lysates containing no dextran sulfate; Lane 5 indicates eluate by boiling SDS loading buffer from the beads bound complex after AP with the biotinylated EGFR aptamers in A431 cell lysates containing 0.01 mM dextran sulfate; Lane 6 indicates eluate by boiling SDS loading buffer from the beads bound complex after AP with the biotinylated EGFR aptamers in A431 cell lysates containing 0.1 mM dextran sulfate; and Lane 7 indicates eluate by boiling SDS loading buffer from the beads bound complex after AP with the biotinylated EGFR aptamers in A431 cell lysates containing 1.0 mM dextran sulfate.

EGFR bands were detected and nonspecific protein bands were reduced at an increased concentration of dextran sulfate (FIG. 3). These data indicate that dextran sulfate can reduce nonspecific proteins in AP assays, thereby allowing highly specific interaction between the aptamers and the EGFR.

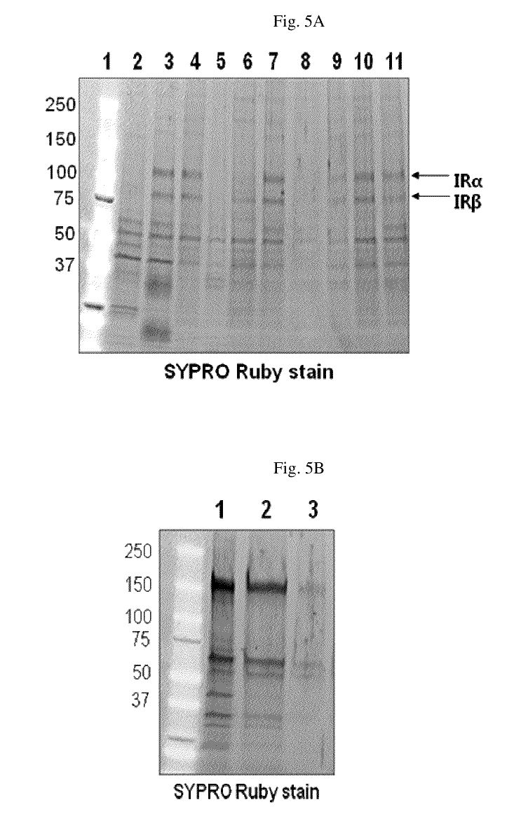

FIG. 4 shows the results of SYPRO Ruby stain showing reduction of nonspecific proteins by using polyanions in AP assay. To reduce background nonspecific proteins encountered in AP assays, the pull-down experiment were conducted using various polyanions as a competitor. Methods were the same as above. Briefly, HEK293 cell lysates were clarified by centrifugation at 12,000×g for 10 min after brief sonication. The lysates were mixed with 10 pmol purified recombinant EGFR, 20 pmol biotinylated EGFR aptamer (#1193-50), and various polyanions. The mixed solution was further incubated with 10 μl Streptavidin magnetic beads. After washing the beads, the bound complex was eluted by boiling SDS loading buffer. The eluate was loaded on a SDS-PAGE (4-15% gradient gel). Protein bands on gel were directly detected by SYPRO ruby.

The results are shown in FIG. 4, wherein Lane 1 indicates protein marker; Lane 2 indicates purified recombinant EGFR proteins (0.5 μg); Lane 3 indicates HEK293 whole cell lysate corresponding to 20 μg of total cellular proteins; Lane 4 indicates eluate by boiling SDS loading buffer from streptavidin magnetic beads after AP with the streptavidin beads; Lane 5 indicates eluate by boiling SDS loading buffer from the beads bound complex after AP with the biotinylated EGFR aptamers/streptavidin beads in HEK293 cell lysates containing 1.0 mM dextran sulfate; Lane 6 indicates eluate by boiling SDS loading buffer from the beads bound complex after AP with the biotinylated EGFR aptamers/streptavidin beads in HEK293 cell lysates containing 1% carboxymethyl-Dextran; Lane 7 indicates eluate by boiling SDS loading buffer from the beads bound complex after AP with the biotinylated EGFR aptamers/streptavidin beads in HEK293 cell lysates containing 1% heparin sodium salt; Lane 8 indicates eluate by boiling SDS loading buffer from the beads bound complex after AP with the biotinylated EGFR aptamers/streptavidin beads in HEK293 cell lysates containing 1.0 mM aurintricarboxylic acid ammonium salt; and Lane 9 indicates eluate by boiling SDS loading buffer from the beads bound complex after AP with the biotinylated EGFR aptamers/streptavidin beads in HEK293 cell lysates containing 1.0 mM suramin sodium salt.

EGFR bands were detected and nonspecific protein bands were reduced by all the polyanions tested. These data indicate that polyanion can reduce nonspecific proteins in AP assays, thereby allowing highly specific interaction between the aptamers and the EGFR (FIG. 4).

### Example 8

### Determination of Elution Condition in Co-AP and AP Assay

### 8.1: Effect of EDTA

AP assay was performed to determine the specific elution towards target proteins in Rat-1/IR cells. Rat-1/IR cell lysates were mixed with the IR aptamers (IR #1652-36)-magnetic agarose beads. More specifically, Rat-1/IR cell lysates corresponding to 100 μg of total cellular proteins that express the insulin receptor (IR) were mixed with 100 pmol IR aptamers (IR #1652-36)-magnetic agarose beads and 1.0 mM dextran sulfate. After washing the beads, the bound complex was eluted by several different eluting buffer. The eluate was loaded on a SDS-PAGE (4-15% gradient gel). Protein bands on gel were directly detected by SYPRO ruby.

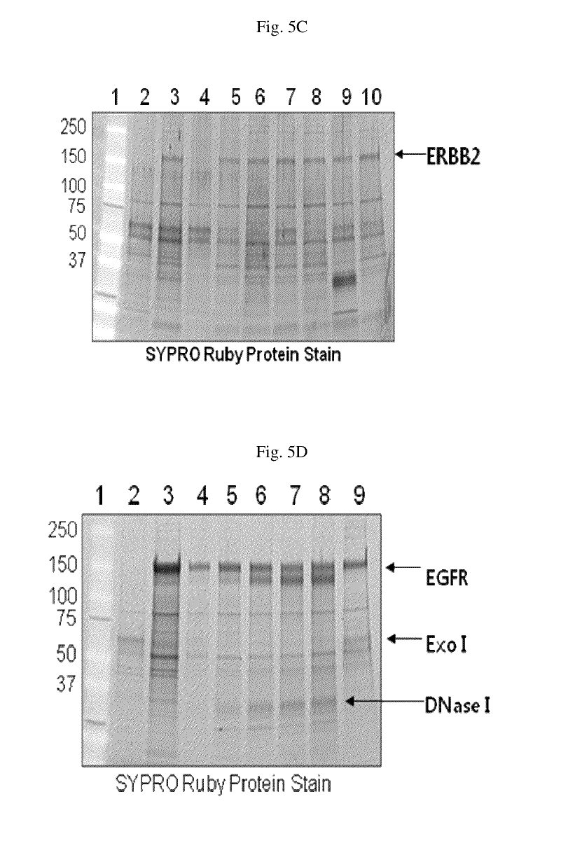

The results are shown in FIG. 5A, wherein Lane 1, protein marker; Lane 2, eluate by boiling SDS loading buffer from beads after AP with blank magnetic agarose beads; Lane 3, eluate by boiling SDS loading buffer from beads after AP with 100 pmol IR aptamers-magnetic agarose beads; Lane 4, eluate by 400 pmol of IR aptamer dissolved in buffer (40 mM HEPES, pH 8.0, 100 mM NaCl) for 1 hr at 60° C.; Lane 5, eluate by 10 mM EDTA, pH 8.0, 95% formamide for 5 min at 60° C.; Lane 6, eluate by 40 mM HEPES (pH 8.0) buffer containing 20 mM EDTA and 100 mM NaCl for 10 min at 37° C.; Lane 7, eluate by 40 mM HEPES (pH 8.0) buffer containing 20 mM EDTA and 100 mM NaCl for 10 min at 60° C.; Lane 8, eluate by 40 mM HEPES (pH 8.0) buffer containing 100 mM EDTA and 100 mM NaCl for 10 min at 37° C.; Lane 9, eluate by 40 mM HEPES (pH 8.0) buffer containing 20 mM EDTA, 0.5 M urea, and 0.02% Tween for 10 min at 37° C.; Lane 10, eluate by 40 mM HEPES (pH 8.0) buffer containing 20 mM EDTA, 0.5 M urea, and 0.02% Tween for 10 min at 60° C.; Lane 11, eluate by 40 mM HEPES (pH 8.0) buffer containing 20 mM EDTA, 3.5 M urea, and 0.02% Tween for 10 min at 60° C.

As shown in FIG. 5A, IR proteins were specifically eluted by IR aptamers as a competitor from the beads bound complex or 20 mM EDTA at high temperature. These data indicate that EDTA can be used for elution of protein complex from aptamer bound protein complex.

AP assay was performed to determine the specific elution towards target proteins in A431 cells. A431 cell lysates were mixed with the EGFR aptamers (#1193-50)-magnetic agarose beads. More specifically, A431 cell lysates corresponding to 1 mg of total cellular proteins that express the EGFR were mixed with 150 pmol EGFR aptamers (#1193-50)-magnetic agarose beads and 1.0 mM dextran sulfate. After washing the beads, the bound complex was eluted by 20 mM EDTA at 60° C. The eluate was loaded on a SDS-PAGE (4-15% gradient gel). Protein bands on gel were directly detected by SYPRO ruby.

The results are shown in FIG. 5B, wherein Lane 1, protein marker; Lane 2, eluate by boiling SDS loading buffer from beads after AP with EGFR-magnetic agarose beads; Lane 3, eluate by 40 mM HEPES (pH 8.0) buffer containing 20 mM EDTA and 100 mM NaCl for 10 min at 60° C.; Lane 4, eluate by 40 mM HEPES (pH 8.0) buffer containing 20 mM EDTA and 100 mM NaCl for 10 min at room temperature;

As shown in FIG. 5B, EGFR proteins were specifically eluted with high yield by 20 mM EDTA at 60° C. from the beads bound complex. These data indicate that EDTA can be used for elution of protein complex from aptamer bound protein complex.

### 8.2: Effect of Free Aptamer (Non-Conjugated Target-Specific Aptamers)

In order to confirm that free aptamers can be used for elution of target proteins as a competitor, Elution test with free aptamers was performed. More specifically, MCF7/ErbB2 cell lysates corresponding to 100 μg of total cellular proteins that express the ErbB2 proteins were mixed with 100 pmol ErbB2 aptamers-magnetic agarose beads. After washing the beads, the bound complex was eluted by free ErbB2 aptamers with different concentration. The eluate was loaded on a SDS-PAGE (4-15% gradient gel). Protein bands on gel were directly detected by SYPRO ruby.

The results are shown in FIG. 5C, wherein Lane 1, protein marker; Lane 2, sample by boiling SDS loading buffer from beads after AP with blank magnetic agarose beads; Lane 3, sample by boiling SDS loading buffer from beads after AP with 100 pmol ErbB2 aptamers-magnetic agarose beads; Lane 4, sample by boiling SDS loading buffer from beads after AP with blank magnetic agarose beads and elution with buffer (40 mM HEPES, pH 8.0, 100 mM NaCl) for 3 hr at 37° C.; Lane 5, eluate by boiling SDS loading buffer after AP with 100 pmol ErbB2 aptamers-magnetic agarose beads and elution with 100 pmol free ErbB2 aptamer dissolved in buffer (40 mM HEPES, pH 8.0, 100 mM NaCl) for 3 hr at 37° C.; Lane 6, eluate by boiling SDS loading buffer after AP with 100 pmol ErbB2 aptamers-magnetic agarose beads and elution with 200 pmol free ErbB2 aptamer dissolved in buffer (40 mM HEPES, pH 8.0, 100 mM NaCl) for 3 hr at 37° C.; Lane 7, eluate by boiling SDS loading buffer after AP with 100 pmol ErbB2 aptamers-magnetic agarose beads and elution with 500 pmol free ErbB2 aptamer dissolved in buffer (40 mM HEPES, pH 8.0, 100 mM NaCl) for 3 hr at 37° C.; Lane 8, eluate by boiling SDS loading buffer after AP with 100 pmol ErbB2 aptamers-magnetic agarose beads and elution with 100 pmol free ErbB2 aptamer dissolved in buffer (40 mM HEPES, pH 8.0, 100 mM NaCl) for 1 hr at 37° C.; Lane 9, eluate by boiling SDS loading buffer after AP with 100 pmol ErbB2 aptamers-magnetic agarose beads and elution with 500 pmol free ErbB2 aptamer dissolved in buffer (40 mM HEPES, pH 8.0, 100 mM NaCl) for 1 hr at 37° C.; Lane 10, eluate by boiling SDS loading buffer after AP with 100 pmol ErbB2 aptamers-magnetic agarose beads and elution with 500 pmol free ErbB2 aptamer dissolved in buffer (40 mM HEPES, pH 8.0, 100 mM NaCl) for 5 hr at 37° C.

As shown in FIG. 5C, ErbB2 proteins were specifically eluted by buffer containing free ErbB2 aptamers from aptamer-magnetic agarose beads bound target proteins.

### 8.3: Effect of DNase

In order to elute target protein complex from beads-aptamers bound protein complex, AP assay with EGFR aptamers was performed. A431 cell lysates corresponding to 100 μg of total cellular proteins that express the EGFR proteins were mixed with 100 pmol EGFR aptamers-magnetic agarose beads conjugate. After washing the beads, the bound complex was eluted by DNase with different concentrations. The eluate was loaded on a SDS-PAGE (4-15% gradient gel). Protein bands on gel were directly detected by SYPRO ruby.

The results are shown in FIG. 5D, wherein Lane 1, protein marker; Lane 2, sample by boiling SDS loading buffer from beads after AP with non-conjugated magnetic agarose beads; Lane 3, sample by boiling SDS loading buffer from beads after AP with 100 pmol EGFR aptamers-magnetic agarose beads; Lane 4, eluate by boiling SDS loading buffer after AP with 100 pmol EGFR aptamers-magnetic agarose beads followed by elution with 0.1 U of DNase (Epicentre, Cat no. D9902K) dissolved in buffer (10 mM Tris, pH 7.5, 2.5 mM MgCl₂, 0.5 mM CaCl₂) for 1 hr at 37° C.; Lane 5, eluate by boiling SDS loading buffer after AP with 100 pmol EGFR aptamers-magnetic agarose beads followed by elution with 0.5 U of DNase (Epicentre, Cat no. D9902K) in buffer (10 mM Tris, pH 7.5, 2.5 mM MgCl₂, 0.5 mM CaCl₂) for 1 hr at 37° C.; Lane 6, eluate by boiling SDS loading buffer after AP with 100 pmol EGFR aptamers-magnetic agarose beads followed by elution with 1 U of DNase (Epicentre, Cat no. D9902K) in buffer (10 mM Tris, pH 7.5, 2.5 mM MgCl₂, 0.5 mM CaCl₂) for 1 hr at 37° C.; Lane 7, eluate by boiling SDS loading buffer after AP with 100 pmol EGFR aptamers-magnetic agarose beads followed by elution with 1 U of DNase (Epicentre, Cat no. D9902K) in buffer (10 mM Tris, pH 7.5, 2.5 mM MgCl₂, 0.5 mM CaCl₂) for 2 hr at 37° C.; Lane 8, eluate by boiling SDS loading buffer after AP with 100 pmol EGFR aptamers-magnetic agarose beads followed by elution with 1 U of DNase (Epicentre, Cat no. D9902K) in buffer (10 mM Tris, pH 7.5, 2.5 mM MgCl₂, 0.5 mM CaCl₂) for 4 hr at 37° C.; Lane 9, eluate by boiling SDS loading buffer after AP with 100 pmol EGFR aptamers-magnetic agarose beads followed by elution with 1 U of Exonuclease I (NEB, Cat no. M0293) in buffer (67 mM Glycine-KOH, pH 9.5, 6.7 mM MgCl₂, 10 mM beta-mercaptoethanol) for 1 hr at 37° C.

As shown in FIG. 5D, EGFR proteins were specifically eluted by both DNase I (endotype nuclease) and Exonuclease I. These data indicate that small amount of DNase and short incubation time is enough for eluting protein complex from aptamer bound protein complex.

### Example 9

### Protein Sequencing Using Mass Spectrometry (MS)

The lyophilized eluants were resuspended and incubated in 50 mM NH₄HCO₃ containing 10 mM dithiothreitol (pH 7.8) for 30 min at 55° C. At the end of the incubation, free thiol groups were alkylated with 40 mM of iodoacetamide in the dark at room temperature for 25 min. Then, tryptic digestion was performed by treating the samples in the buffer containing 50 mM ammonium bicarbonate, 5 mM CaCl₂, and 10 μg/ml trypsin at 37° C. for 12-16 h followed by lyophilization. The lyophilized peptide mixtures were solubilized in 0.1% formic acid and loaded onto a microcapillary column packed with C18 RP resin in 75 μm silica tubing (8 μm inner diameter of the orifice, 10 cm. in length). For the elution of peptides, buffers A (0.1% formic acid) and B (80% acetonitrile containing 0.1% formic acid) were prepared and eluted with 5% of the buffer B for 25 min, 20% for 5 min, 60% for 50 min, and 100% for 5 min at a flow rate of 300 nL/min. Then, the column was equilibrated with 5% of the buffer B for 15 min prior to the next running. For the identification of peptide, the eluted peptides were subjected to mass (MS) spectrometry using 7-tesla Finnigan LTQ-FT MS spectrometer (Thermo Electron, Bremen, Germany) equipped with a nano-ESI source in positive ion mode at the spray voltage of 2.5 kV. MS and MS/MS spectra were obtained with the capillary heated to 220° C., an ESI voltage of 2.5 kV, 35% of collision energy, and 1 Da of isolation width. The full scan was performed at a resolution of 100,000 FWHM (the full width at half maximum) intensity, and then data-dependent MS/MS analysis was performed from the three most abundant MS ions. The spectra were analyzed with Mascot Daemon (Matrix Science, London, UK) using the IPI human database (IPI.HUMAN.v.3.72). Peptides were considered to be identified at the peptide tolerance of ±50 ppm, fragment mass tolerance of ±0.8 Da, two missed trypsin cleavage, oxidation of Met, and fixed modification of carbamidomethyl cysteine. Peptide score is −10×Log(P), in which P signifies the probability that the observed match is a random event. Individual peptide scores over 35 are considered as identity or extensive homology (P<0.05).

### Example 10

### Protein Data Analysis

Data obtained from mass spectrometry were searched using Mascot (Matrix Sciences, London, UK) as the search engine. All searches were performed against the NCBInr protein sequence database. The Mascot searching was performed using the default settings for the specific instrument type as supplied by Matrix Sciences.

### Example 11

### Identification of Physiologically Relevant Protein-Protein Interactions by Using Insulin Receptor-Specific Aptamers

Insulin mediates cell signaling through activation with the insulin receptor (IR), a tyrosine kinase receptor. Insulin receptor is a transmembrane receptor that is activated by insulin. Two alpha subunits and two beta subunits make up the insulin receptor. The beta subunits pass through the cellular membrane and are linked by disulfide bonds. The activated IRkinase transduces the insulin signal by activating pathways such as the Ras-Raf-MEK-ERK, the PI3K-PDK-AKT, the c-Cbl-Glut4, the PI3K-Rab4-Glut4 and the PI3K-Rac-MEKK1-MKK4-JNK pathways. These pathways are modulated by complex networks of signaling inputs. The balancing of signals that transit the pathways stimulated by insulin provide the specific cell response to insulin signaling. Insulin signaling is mediated by cascades of phosphorylation/dephosphorylation events, guanine nucleotide exchange events and spatial positioning of signaling, scaffolding and adaptor molecules. Molecules that bind directly to the IR such as the IRS family, SHC, PI3K and GRB2 transduce the insulin signal into the appropriate pathways.

Insulin stimulates the uptake of glucose primarily in adipocytes and myocytes by the translocation of the glucose channel, GLUT4, from the insulin responsive component (IRC) to the cell surface by two pathways, PI3K-AKT-atypical PKClamda-Rab4 and c-Cbl-APS-CAP. It modulates the growth/differentiation response via the Raf-MEK-ERK axis of the p21/Ras pathway. Protein synthesis, glycogenesis, lipogenesis and anti-apoptotic effects are controlled primarily via the PI3K-PDK-Akt/PKB pathway. The specific pathways require down-stream effectors such as GSK3, mTor, p70S6 kinase and pro-apoptotic factors Bad and FKHRL-1.

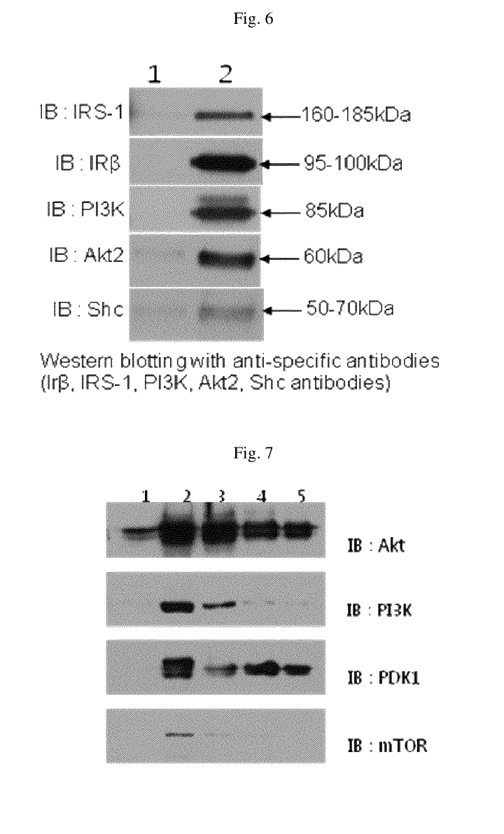

To check the performance of aptamers in Co-AP assays, Co-AP experiments were performed using IR aptamer. Methods were the same as above. Briefly, Rat-1/IR cells were starved to serum-free medium overnight (16-18 h) and were stimulated with 100 nM insulin. Whole cell lysates from Rat-1/IR cell lines that express the IR were clarified by lysis buffer and centrifugation at 12,000×g for 10 min. The cleared lysates were incubated with 500 pmoles IR-aptamers (IR #1652-36)-magnetic agarose beads. The mixed solution was washed four times with detergent free lysis buffer. The bound complex was eluted by 20 mM EDTA in 40 mM HEPES containing 0.1M NaCl. To identify IR interacting proteins, the aptoprecipitation protein complex can then be subjected to SDS-PAGE and Western blotting analysis was performed (FIG. 6). Membranes blotted were probed with specific antibodies [anti IR β Ab (Cell signaling #3025) for IR β, anti IRS1 Ab (Millipore #06-248) for IRS1, anti Akt Ab (Cell signaling #4691) for Akt, anti PI3K Ab (Santa cruz #sc-1637) for PI3K, anti Shc Abs (Cell signaling #2432) for Shc].

The results are shown in FIG. 6, wherein Lanes 1 indicate eluate by 20 mM EDTA buffer from the beads bound complex after AP with the magnetic agarose beads; Lane 2 eluate by 20 mM EDTA buffer from the beads bound complex after AP with the IR aptamers-magnetic agarose beads.

As a result, IRβ was clearly detected in Co-AP eluted proteins from total proteins bound to bead, while IRβ protein was not detected in blank bead. IR interacting proteins such as IRS-1 and PI3K were identified in aptamer based Co-AP assay, while any of IR interacting proteins were not clearly detected in blank beads. Interestingly, Akt2 and Shc protein involved in IR signal pathway were also identified in Co-AP assay. These results indicated that IR aptamer based Co-AP assay can be a useful tool for the identification of physiologically relevant IR protein-protein interactions.

### Example 12

### Identification of Physiologically Relevant Protein-Protein Interactions by Using AKT2-Specific Aptamers

In humans, there are three genes in the “Akt family”: Akt1, Akt2, and Akt3. These genes code for enzymes that are members of the non-specific serine/threonine-protein kinase family.

Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt1 was originally identified as the oncogene in the transforming retrovirus, AKT8. Akt2 is an important signaling molecule in the insulin signaling pathway. It is required to induce glucose transport. In a mouse which is null for Akt1 but normal for Akt2, glucose homeostasis is unperturbed, but the size of animals are smaller than normal, consistent with a role for Akt1 in growth. In contrast, mice which do not have Akt2, but have normal Akt1, have mild growth deficiency and display a diabetic phenotype (insulin resistance), again consistent with the idea that Akt2 is more specific for the insulin receptor signaling pathway. The role of Akt3 is less clear, though it appears to be predominantly expressed in brain. It has been reported that mice lacking Akt3 have smaller brain size.

Many cell surface receptors induce the production of second messengers that activate phosphoinositide 3-kinase (PI3K). Akt is located downstream of PI3K and, therefore, functions as a part of Wortmannin-sensitive signaling pathway. PI3K generates phosphorylated phosphatidylinositides (PI-3,4-P₂ and PI-3,4,5-P₃) in the cell membrane which bind to the amino-terminal pleckstrin homology (PH) domain of Akt. PI-3,4-P₂ and PI-3,4,5-P₃ also activate phosphoinositide-dependent kinase (PDK) which phosphorylates Thr³⁰⁸ of membrane-bound Akt. Akt can then be phosphorylated by its activating kinases, phosphoinositide dependent kinase 1 (PDPK1 at threonine 308) and mTORC2 (at serine 473).

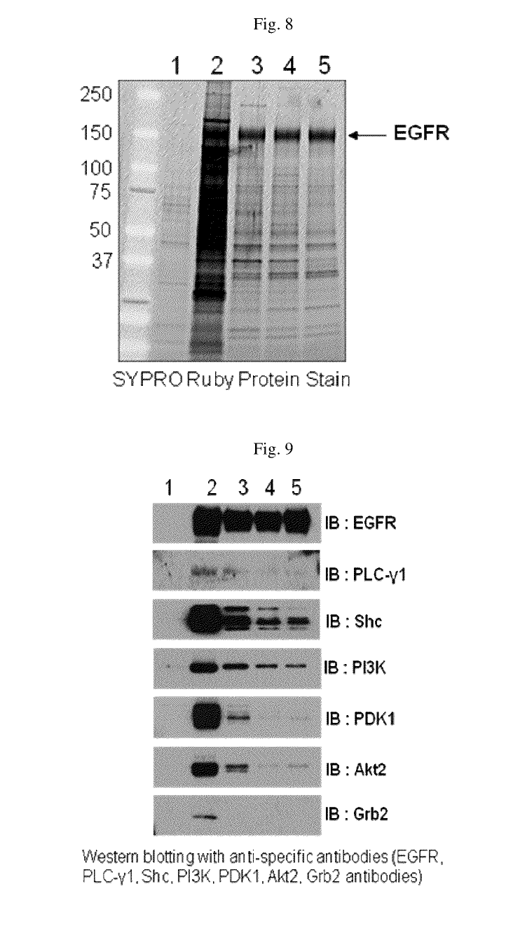

A western blotting analysis was performed to study DxSO₄ effects in Co-AP assays with various DxSO₄ concentrations in a binding buffer. Methods were the same as described above. Briefly, MCF7 cells were starved to serum-free medium overnight (16-18 h) and were stimulated with 100 nM insulin. Whole cell lysates from MCF7 cell lines that express the AKT2 were clarified by lysis buffer and centrifugation at 12,000×g for 10 min. The cleared lysates were incubated with 500 pmoles AKT2-aptamers (AKT #2153-02-02)-magnetic agarose beads and 0, 0.01 mM, 0.1 mM, or 1 mM DxSO₄. The mixed solution was washed four times with lysis buffer. The bound complex was eluted by 20 mM EDTA in 40 mM HEPES containing 0.1M NaCl. To identify AKT2 interacting proteins, the aptoprecipitated protein complex then was subjected to SDS-PAGE, and Western blotting analysis was performed (FIG. 7). Membranes blotted were probed with specific antibodies [anti akt Ab (Cell signaling #4691), anti PI3K Ab (Santa cruz sc-1637), anti PDK1 Ab (Cell signaling #3062), and anti mTOR Ab (Cell signaling #2972)].

The results are shown in FIG. 7, wherein Lane 1 indicates an eluation by 20 mM EDTA buffer from the beads bound complex after aptoprecipitation (AP) with the magnetic agarose beads; Lane 2 indicates an eluation by 20 mM EDTA buffer from the beads bound complex after AP with the AKT2 aptamers-magnetic agarose beads in the absence of DxSO₄; Lane 3 indicates an eluation by 20 mM EDTA buffer from the beads bound complex after AP with the AKT2 aptamers-magnetic agarose beads in the presence of 0.01 mM DxSO₄; Lane 4 indicates an eluation by 20 mM EDTA buffer from the beads bound complex after AP with the AKT2 aptamers-magnetic agarose beads in the presence of 0.1 mM DxSO₄; Lane 5 indicates an eluation by 20 mM EDTA buffer from the beads bound complex after AP with the AKT2 aptamers-magnetic agarose beads in the presence of 1 mM DxSO₄.

The results showed that AKT interacting proteins such as Akt, PI3K, PDK1 and mTOR were identified in aptamer based Co-AP assay, while any of AKT interacting proteins were not detected in control condition (magnetic agarose beads). These results indicated that aptamer based Co-AP assay can be a useful tool for the identification of physiologically relevant protein-protein interactions study.

### Example 13

### Identification of Physiologically Relevant Protein-Protein Interactions by Using EGFR-Specific Aptamers

The epidermal growth factor (EGF) receptor is a transmembrane tyrosine kinase that belongs to the HER/ErbB protein family. Ligand binding results in receptor dimerization, autophosphorylation, activation of downstream signaling, internalization, and lysosomal degradation. Phosphorylation of EGFR at Tyr845 in the kinase domain is implicated in stabilizing the activation loop, maintaining the active state enzyme, and providing a binding surface for substrate proteins. c-Src is involved in phosphorylation of EGFR at Tyr845. The SH2 domain of PLCγ binds at phospho-Tyr992, resulting in activation of PLCγ-mediated downstream signaling. Phosphorylation of EGFR at Tyr1045 creates a major docking site for c-Cbl, an adaptor protein that leads to receptor ubiquitination and degradation following EGFR activation. The GRB2 adaptor protein binds activated EGFR at phospho-Tyr1068. A pair of phosphorylated EGFR residues (Tyr1148 and Tyr1173) provides a docking site for the Shc scaffold protein, with both sites involved in MAP kinase signaling activation. Phosphorylation of EGFR at specific serine and threonine residues attenuates EGFR kinase activity. EGFR carboxy-terminal residues Ser1046 and Ser1047 are phosphorylated by CaM kinase II; mutation of either of these serines results in upregulated EGFR tyrosine autophosphorylation.

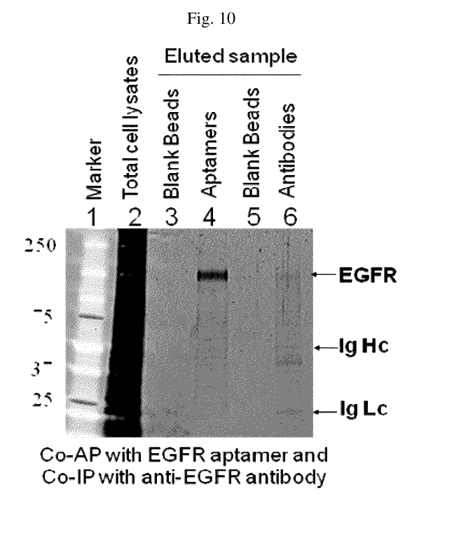

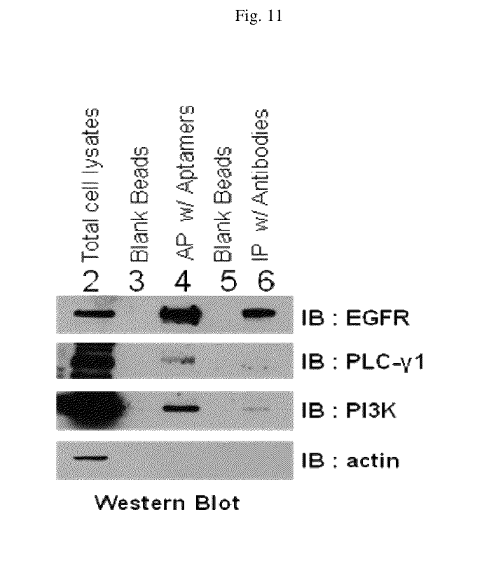

In order to study additional DxSO₄ effects in Co-AP assays, Co-AP experiments were performed with various DxSO₄ concentrations in a binding buffer. Methods were the same as described above. Briefly, A431 cells were starved to serum-free medium overnight (16-18 h) and were stimulated with 100 nM EGF. Whole cell lysates from A431 cell lines that express the EGFR were clarified by lysis buffer and subjected to centrifugation at 12,000×g for 10 min. The cleared lysates were incubated with 500 pmoles EGFR-aptamers (#1193-50)-magnetic agarose beads and 0, 0.01 mM, 0.1 mM, or 1 mM DxSO₄. The mixed solution was washed four times with a lysis buffer. The bound complex was eluted by 20 mM EDTA in 40 mM HEPES containing 0.1M NaCl. To identify EGFR interacting proteins, the aptoprecipitated protein complex then was subjected to SDS-PAGE (FIG. 8) followed by Western blotting analysis (FIG. 9). Membranes blotted were probed with specific antibodies [anti EGFR Ab (Cell signaling #2232), anti PLC-γ1 Ab (R&D systems #MAB3288), anti Shc Abs (Cell signaling #2432), anti PI3K Ab (Santa cruz #sc-1637), anti Akt Ab (Cell signaling #4691), anti PDK1 Ab (Cell signaling #3062), and anti-Grb2 Abs (R&D systems #MAB3846).

The results are shown in FIG. 8 (SDS-PAGE) and FIG. 9 (Western blot), wherein Lane 1 indicates an eluation by 20 mM EDTA buffer from the beads bound complex after AP with the magnetic agarose beads; Lane 2 indicates an eluation by 20 mM EDTA buffer from the beads bound complex after AP with the EGFR aptamers-magnetic agarose beads in the absence of DxSO₄; Lane 3 indicates an eluation by 20 mM EDTA buffer from the beads bound complex after AP with the EGFR aptamers-magnetic agarose beads in the presence of 0.01 mM DxSO₄; Lane 4 indicates an eluation by 20 mM EDTA buffer from the beads bound complex after AP with the EGFR aptamers-magnetic agarose beads in the presence of 0.1 mM DxSO₄; Lane 5 indicates an eluation by 20 mM EDTA buffer from the beads bound complex after AP with the EGFR aptamers-magnetic agarose beads in the presence of 1 mM DxSO₄.

As shown in FIG. 8, nonspecific protein bands were reduced at an increased concentration of DxSO₄, while EGFR receptor bands were not decreased. As shown in FIG. 9, EGFR interacting proteins such as PLC-γ1, Shc, PI3K, PDK1, Grb2 were identified in aptamer based Co-AP assay, while none of EGFR interacting proteins was detected in control condition (non-conjugated-magnetic agarose beads). These results indicated that aptamer based Co-AP assay can be a useful tool for the identification of physiologically relevant protein-protein interactions study.

### Example 14

### Comparison of Performance of Aptamer and Antibody (Ab) in Co-AP Assay

To compare the performance of aptamers and antibodies in Co-AP assays, Co-AP experiments were performed. Subconfluent A431 cell culture was starved overnight and stimulated with 100 nM EGF. Cell extracts were prepared from EGFR expressing human squamous carcinoma cell line A431. The extracts were incubated with anti-EGFR aptamers (75 pmol) and 0.01 mM DxSO₄ or for Co-AP assay and were incubated with anti-EGFR antibody (75 pmol) for Co-AP assay. After incubation, the unbound proteins were washed away and the bound proteins were eluted. The eluted samples were separated with SDS-PAGE and stained with SYPRO ruby stain (4-15% gradient gel). The eluted samples were also blotted onto a PVDF membrane and probed with specific antibodies [anti EGFR Ab (signaling, #2232), anti PLC-γ1 Ab (R&D systems, #MAB3288), and anti PI3K Ab (Santa cruz, #sc-1637)]. Co-IP using antibody was performed according to the manufacturer's instruction.

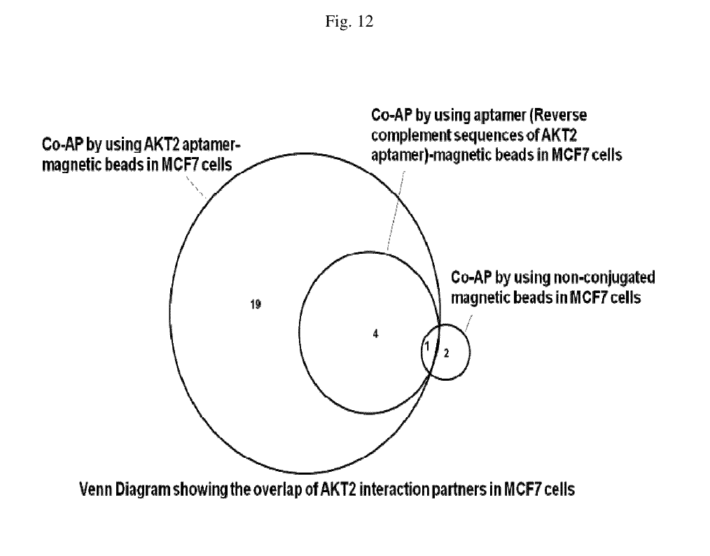

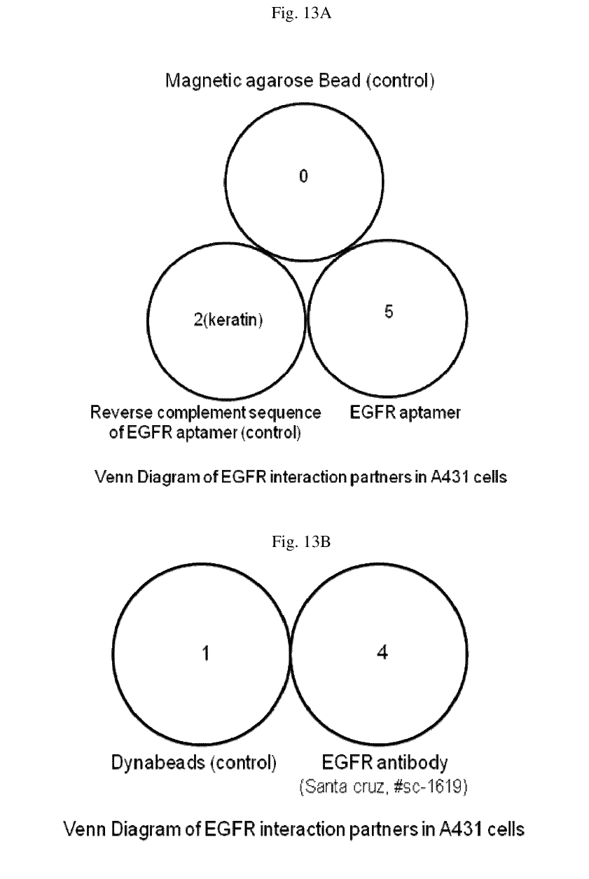

The results are shown in FIG. 10 (SDS-PAGE) and FIG. 11 (Western blot), wherein Lane 1 indicates protein marker; Lane 2 indicates A431 whole cell lysate corresponding to 20 μg of total cellular proteins; Lanes 3, eluate by boiling SDS loading buffer from beads after AP with blank magnetic agarose beads followed by elution with 20 mM EDTA buffer; Lanes 4, eluate by boiling SDS loading buffer from beads after AP with 75 pmol EGFR aptamers-magnetic agarose (SDS-PAGE) or 500 pmol EGFR aptamers-magnetic agarose beads (Western blot) followed by elution with 20 mM EDTA buffer; Lanes 5, eluate by boiling SDS loading buffer from beads after IP with blank dynabeads followed by elution with low-pH elution according to product instructions; Lanes 6, eluate by boiling SDS loading buffer from beads after IP with 75 pmol EGFR antibody-dynabeads (SDS-PAGE) or 100 pmol EGFR antibody-dynabeads (Western blot) followed by elution with low-pH elution according to product instructions.

As shown in FIGS. 10 and 11, EGFR was clearly detected in Co-AP eluted proteins from total proteins bound to bead, while EGFR protein was not clearly detected in Co-IP eluted proteins from total proteins bound to bead (Left image). EGFR aptamers were highly specific to EGFR, while anti-EGFR Abs precipitated extraordinarily small amounts of EGFR. EGFR interacting proteins such as PLC-γ1 and PI3K were identified in aptamer based Co-AP assay, while none of EGFR interacting proteins was clearly detected in antibody based Co-IP assay (Right image).

Although data are not shown here, other EGFR interacting proteins such as Shc and Grb2 were clearly identified in Co-AP assay. Interestingly, PDK1 and Akt2 protein involved in EGFR signal pathway were also identified (data not shown) in Co-AP assay. These results indicated that EGFR aptamer based Co-AP assay can be a useful tool for the identification of physiologically relevant EGFR protein-protein interactions.

### Example 15

### Proteomic Analysis of the Aptoprecipitated Protein Complex by Using Co-AP Method

Proteins potentially Akt ans EGFR interacting proteins were identified using Co-AP and mass spectrometry-based methodology. It is important to emphasize that no transfection or overexpression of Akt or any other partners were performed here, and therefore this study has the advantage to rely exclusively on physiological levels of protein expression and interaction.

15.1: Identification of AKT2 Protein Complex Partner by Using Co-AP Method and Mass Spectrometry Analysis from MCF7 Cells

In order to confirm performance of aptamer in Co-AP assay and discover new protein interactions in AKT signaling networks in MCF7 cells, Co-AP assay with AKT2 aptamers was performed.

MCF7 cells were starved to serum-free medium overnight (16-18 h) and were stimulated with 100 nM insulin. Whole cell lysates from MCF7 cell lines were clarified by a lysis buffer and subjected to centrifugation at 12,000×g for 10 min. The cleared lysates were incubated with 500 pmoles AKT2-aptamers-magnetic agarose. The mixed solution was washed four times. The bound complex was eluted by 0.5 U DNaseI for 1 hr at 37° C. and inactivate DNaseI by Heating 65° C. for 10 min Subsequently, the eluate was digested by trypsin for mass spectrometry analysis.

Peptide score is −10×Log (P), in which P signifies the probability that the observed match is a random event. Individual peptide scores over 35 are considered as identity or extensive homology (P<0.05). 20 proteins showing protein interactions meeting these criteria are shown in Table 2.

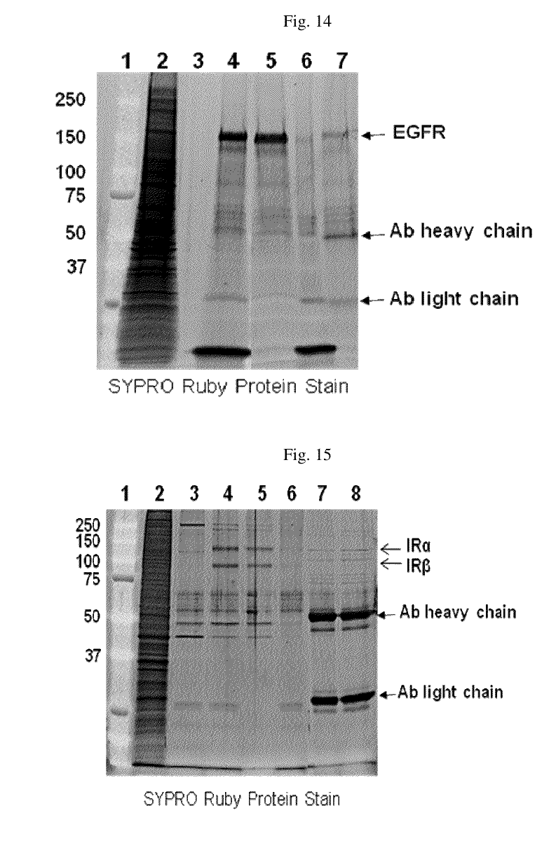

3 proteins were identified in Co-AP by using blank magnetic agarose beads and 5 proteins were identified in Co-AP by using control aptamers (reverse complement sequences of AKT2 aptamers) conjugated to magnetic agarose beads in MCF7 cells, while 24 proteins were identified Co-AP by using AKT2 aptamers-magnetic agarose beads in MCF7 cells. As shown in FIG. 12, Venn diagram shows that 19 proteins identified in Co-AP using AKT2 aptamers were found to interact with AKT2 as well as AKT2 interacting proteins with a high probability score. 19 proteins identified in Co-AP using AKT2 aptamers were listed in Table 2 with Mascot protein score and number of matched peptides.

Mass spectrometry analysis confirmed the presence of several interacting proteins reported as AKT interacting partner (Nucleolin, HSPA8, SYNCRIP, HMGB1, PRMT1, ACLY, ACTN4, HSPA9, KRT18, PRDX1), but also resulted in the identification of many new putative binding partners (LSM2, C14orf156, CNBP, KRT7, KRT7, HMGB3L1, LSM3, RPA1) (complete lists in Table 2).

### 5.2: Literature Analysis of the AKT Protein Complex

The serine/threonine kinase Akt is a key mediator of cell survival and cell growth that is activated by most of growth factors, but its downstream signaling largely remains to be elucidated. To identify signaling partners of Akt by using aptamer based Co-AP method, proteins co-precipitated with Akt aptamers in MCF-7 breast cancer cells were analyzed. Mass spectrometry analysis Akt co-precipitates allowed the identification of 19 proteins: Nucleolin, HSPA8, SYNCRIP, HMGB1, PRMT1, LSM2, C14orf156, AKT1, ACLY, ACTN4, CNBP, AKT2, KRT7, HSPA9, HMGB3L1, KRT18, LSM3, RPA1, PRDX1. The identification of these putative Akt binding partners were identified with AKT2-aptamer-magnetic agarose beads.

Among them, 10 proteins have previously been reported through proteomic studies. Among putative partners of Akt identified in the present study, the nucleolin was found with best score as a putative Akt partner. It has been reported that phosphorylated form of Akt translocates to the nucleus of PC12 cells under nerve growth factor stimulation and associates with the nuclear matrix protein nucleolin (Journal of cellular physiology, 2003, 196, 79-88). This clearly showed that an in vivo interaction of nucleolin and phosphorylated Akt in nuclei of PC 12 cells using an immunoprecipitation (IP) of with anti-nucleolin or anti-p-Akt antibody was detected. Western blotting analysis performed with both antibodies on the same samples showed that antibody to nucleolin was able to co-immunoprecipitate nuclear p-Akt. These findings strongly suggest that the intranuclear translocation of active Akt is an important step in the signaling pathways (Journal of cellular physiology, 2003, 196, 79-88).

Among putative partners of Akt identified in the present study, some of the identified proteins were found to be constitutively associated with Akt since their interactions were not growth factor-regulated. Protein chaperones were identified, such as the heat shock protein HSPA8 and HSPA9 (mortalin). It has been previously reported that HSPA8 is essential for Akt signaling in endothelial function (Arterioscler Thromb Vasc Biol., 2010, 30, 491-497). They have demonstrated that HSPA8 is essential for multiple EC functions via the PI3K/Akt pathway. HSPA8 may promote endothelial health and may, therefore, be important in the treatment of disorders in which there is evidence of endothelial dysfunction. We found HSPA9 (mortalin) as a putative Akt partner. It has been already reported that HSPA9 co-immunoprecipitated with AKT. They analyzed proteins co-immunoprecipitated with Akt in MCF-7 breast cancer cells. Mass spectrometry analysis of SDS-PAGE-separated Akt co-immunoprecipitates allowed the identification of HSPA9 (Molecular & Cellular Proteomics, 2007, 6, 114-124; Proteome Science 2009, 7, 1477-5956).

Another protein identified was ACTN4 (Alpha-actinin-4) as a putative Akt partner. It has been already reported that ACTN4 co-immunoprecipitated with AKT. The major protein band observed in Akt coimmunoprecipitates was found to be the ACTN4 for which a 14-fold increase was observed in Akt-activated condition when compared to a non-activated condition. In addition, proteins co-immunoprecipitated with Akt in MCF-7 breast cancer cells were analyzed. Mass spectrometry analysis of SDS-PAGE-separated Akt co-immunoprecipitates allowed the identification of ACTN4 (Molecular & Cellular Proteomics, 2007, 6, 114-124). The interaction between Akt and ACTN4 was further confirmed by reverse immunoprecipitation, and confocal microscopy demonstrated a co-localization specifically induced under growth factor stimulation. Using a phospho-Akt substrate antibody, the phosphorylation of ACTN4 on an Akt consensus site was detected upon growth factor stimulation, both in cellulo and in vitro, suggesting that actin is a substrate of Akt kinase activity. Together these data suggest the identification of ACTN4 as a new functional target of Akt signaling.

It has been further reported that ACTN4 co-immunoprecipitated with AKT. By using RePCA (A retrovirus-based protein complementation assay), they identified a series of 24 potential interaction partners or substrates of the serine/threonine protein kinase AKT1. They confirm that ACTN4 interacts physically and functionally with AKT1 (PNAS, 2006, 103:41, 15014-15019).

Protein identified in this experiment was PRDX1 as a putative Akt partner. It has been previously reported that PRDX1 co-immunoprecipitated with AKT. By using RePCA (A retrovirus-based protein complementation assay), they identified that PRDX1 interacts physically and functionally with AKT1 (PNAS, 2006, 103:41, 15014-15019).

Protein identified in this experiment was HMGB1 as a putative Akt partner. High-mobility group box 1 (HMGB1) is a DNA-binding protein, which on release from cells exhibits potent inflammatory actions. It has been previously reported that HMGB1 activates downstream signaling pathways involving Akt, ERK1/2, and p38 MAPK in human neutrophils (Am J Physiol Cell Physiol, 2003, 284, 870-879). It has been previously reported that cytokine-induced HMGB1 secretion could be attenuated by inhibitors of MEK1/MEK2, protein kinase C, and PI-3 kinase/Akt, suggesting that such agents might be useful in attenuating HMGB1-mediated inflammation (Arterioscler Thromb Vasc Biol. 2004, 24, 2320-2325). It has been also previously reported that Akt and p38 MAPK contribute to the HMGB1-induced NAD(P)H oxidase activation. They tested the role of Akt and p38 MAPK in HMGB1-induced NAD(P)H oxidase activation. They detected Akt and p38 MAPK activation in PMN in response to HMGB1 using kinase assays (The Journal of Immunology, 2007, 178, 6573-6580)

Protein identified in this experiment was PRMT1 as a putative Akt partner. Yamagata et al. (Mol. Cell, 2008, 32, 221-231) demonstrate that FOXO methylation by PRMT1 prevents Akt-mediated phosphorylation and enhances FOXO1-dependent transcription. They identified that a role for the protein methyltransferase, PRMT1, in Akt-dependent regulation of the FOXO1 transcription factor and demonstrate another level of control in this critical metabolic and cell survival signaling pathway.

Protein identified in this experiment was ACLY as a putative Akt partner. Enhanced glucose and lipid metabolism is one of the most common properties of malignant cells. ATP citrate lyase (ACLY) is a key enzyme of de novo fatty acid synthesis responsible for generating cytosolic acetyl-CoA and oxaloacetate. Toshiro et al. (Cancer Res 2008, 68:20, 8547-8554) analyzed ACLY expression in a subset of human lung adenocarcinoma cell lines and showed a relationship with the phosphatidyl-inositol-3 kinase-Akt pathway. The introduction of constitutively active Akt into cells enhanced the phosphorylation of ACLY, whereas dominant-negative Akt caused attenuation. ACLY phosphorylation is directly regulated by PI3K-Akt pathway. They determined whether ACLY is a downstream target of the PI3K-Akt pathway in lung cancer. It has been also found that Akt directly phosphorylates and activates ACLY (J Biol Chem, 2002, 277:33895-33900).

Protein identified in this experiment was Keratin 18 as a putative Akt partner. Keratin 18 is simple epithelial intermediate filament (IF) proteins, whose expression is differentiation- and tissue-specific, and is maintained during tumorigenesis. Vimentin IF is often coexpressed with keratins in cancer cells. Recently, IF have been proposed to be involved in signaling pathways regulating cell growth, death and motility. The PI3K/Akt pathway plays a pivotal role in these processes. Anne-Marie Fortier et al. (FEBS Lett. 2010, 5; 584, 984-988) investigated the role of Akt (1 and 2) in regulating IF expression in different epithelial cancer cell lines. It has been also previously reported that the enhanced apoptosis in the livers of mice that express K18-Gly-involves the inactivation of Akt1 and protein kinase CO as a result of their site-specific hypophosphorylation. Akt1 binds to K8, which probably contributes to the reciprocal hyperglycosylation and hypophosphorylation of Akt1 that occurs on K18 hypoglycosylation, and leads to decreased Akt1 kinase activity. Therefore, K18 glycosylation provides a unique protective role in epithelial injury by promoting the phosphorylation and activation of cell-survival kinases (Nature cell biology, 2010, 12: 9, 876-886).

Protein identified in this experiment was SYNCRIP (Isoform 1 of Heterogeneous nuclear ribonucleoprotein Q) as a putative Akt partner. YB-1 is a broad-specificity RNA-binding protein that is involved in regulation of mRNA transcription, splicing, translation, and stability. It has been previously reported that Akt is associated with inactive mRNPs including SYNCRIP protein and that activated Akt may relieve translational repression of the YB-1-bound mRNAs (Molecular and cellular biology, 2006, 277-292; Cell cycle-2006, 1143-1147).

Mass spectrometry analysis Akt co-precipitates allowed the identification of 19 proteins. Among them, 10 proteins have previously been reported through proteomic studies as described above. 9 proteins which may potentially interact with Akt that are discussed require further experiments to confirm their functional interactions.

In conclusion, the proteomics exploration of Akt signaling in breast cancer cells led to the demonstration that the aptamer based Co-AP method can be a useful tool for the identification of physiologically relevant protein-protein interactions study.

TABLE 2
Proteins identified by using AKT2 aptamer based Co-AP method.

| Protein name | Score | Peptides |
|---|---|---|
| Nucleolin | 270 | 10 |
| HSPA8 | 212 | 7 |
| SYNCRIP | 150 | 3 |
| HMGB1 | 144 | 4 |
| PRMT1 | 139 | 6 |
| LSM2 | 123 | 1 |
| C14orf156 | 108 | 3 |
| AKT1 | 91 | 5 |
| ACLY | 88 | 5 |
| ACTN4 | 76 | 2 |
| CNBP | 73 | 2 |
| AKT2 | 72 | 4 |
| KRT7 | 64 | 2 |
| HSPA9 | 59 | 3 |
| HMGB3L1 | 57 | 1 |
| KRT18 | 51 | 5 |
| LSM3 | 41 | 1 |
| RPA1 | 40 | 1 |
| PRDX1 | 38 | 1 |

15.3: Identification of EGFR Protein Complex Partner by Using Co-AP Method and Mass Spectrometric Analysis from A431 Cells

In order to compare the superior performance of aptamer over the antibodies in Co-precipitation assay, protein interactions in EGFR signaling networks in A431 cells were studied.

A431 cells were starved to serum-free medium overnight (16-18 h) and were stimulated with 100 nM EGF. Whole cell lysates from A431 cell lines that express the EGFR protein were clarified by lysis buffer and subjected to centrifugation at 12,000×g for 10 min. The cleared lysates were incubated with either 500 pmoles EGFR-aptamers-magnetic agarose or 500 pmoles EGFR-antibody-Dynabead M270. The mixed solution was washed four times. The bound complex was eluted by 0.5 U DNaseI for 1 hr at 37° C. and inactivate DNaseI by Heating 65° C. for 10 min. Eluate was digested by trypsin for mass spectrometry analysis.

Peptide score is −10×Log (P), in which P signifies the probability that the observed match is a random event. Individual peptide scoring over 35 is considered as identity or extensive homology (P<0.05).

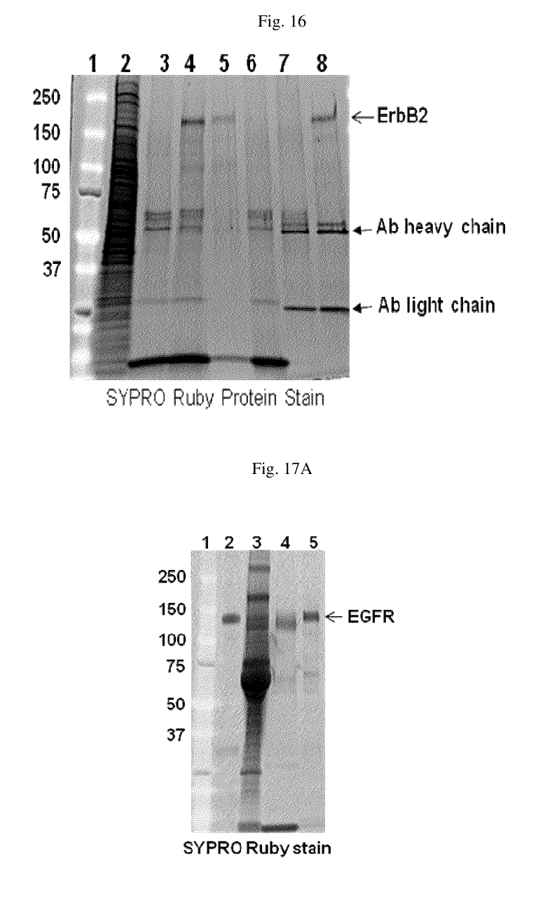

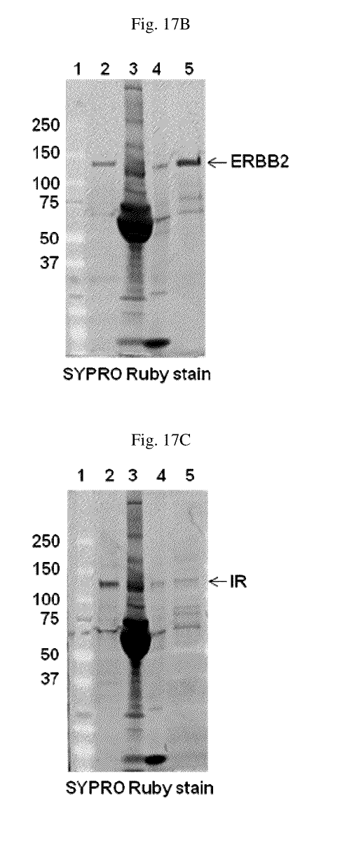

No proteins in Co-AP was identified by using blank magnetic agarose beads, but 2 proteins were identified in Co-AP by using control aptamers (reverse complement sequences of EGFR aptamers)-magnetic agarose beads in A431 cells, while 5 proteins were identified in Co-AP by using EGFR aptamers-magnetic agarose beads in A431 cells. As shown in FIG. 13a, Venn diagram shows that 5 proteins identified in Co-AP using EGFR aptamers were found to interact with EGFR interacting proteins with a high probability score. 5 proteins identified in Co-AP using EGFR aptamers were listed in Table 3 with Mascot protein score and number of matched peptides.

As shown in FIG. 13b, 1 protein in Co-IP was identified by using blank Dynabeads, while 4 proteins were identified in Co-IP by using antibody-Dynabeads in A431 cells. 4 proteins identified in Co-IP using EGFR antibodies were listed in Table 3 with Mascot protein score and number of matched peptides.

Proteins identified by using EGFR aptamer confirmed the presence of several interacting proteins reported EGFR interaction partner (calmodulin, DECR1, G3BP1), but also resulted in the identification of new putative binding partners (ACLY, MYL6B) (complete lists in Table 3). In case of proteins identified by using EGFR antibody, only ERBB4 was reported as an EGFR interacting partner.

TABLE 3
Proteins identified by using EGFR aptamer and antibody.

| | | |
|---|---|---|
| Protein name | Score | Peptides |

Proteins identified by using EGFR aptamer

| | | |
|---|---|---|
| EGFR | 148 | 11 |
| Calmodulin | 108 | 1 |
| DECR1 | 98 | 3 |
| G3BP1 | 84 | 2 |
| ACLY | 57 | 4 |
| MYL6B | 52 | 3 |

Proteins identified by using EGFR antibody

| | | |
|---|---|---|
| EGFR | 405 | 17 |
| SERPINH1 | 134 | 3 |
| KRT1 | 98 | 3 |
| Myosin-reactive | 88 | 1 |
| immunoglobulin light chain | | |
| variable region | | |
| ERBB4 | 81 | 5 |

### 15.4: Literature Analysis of the EGFR Protein Complex

To identify signaling partners of EGFR, proteins co-precipitated by using either aptamer based Co-AP method or antibody based Co-IP method in A431 cells were analyzed. Mass spectrometry analysis of EGFR co-precipitates allowed the identification of 5 proteins: calmodulin, DECR1, G3BP1, ACLY, MYL6B. These putative EGFR binding partners were identified only when EGFR-aptamer-magnetic agarose beads were used.

Among putative partners of EGFR identified in the present study, the calmodulin was found with best score as a putative EGFR partner. It has been reported that Calmodulin binds to the EGFR Juxtamembrane Domain with an apparent dissociation constant (Kd) of approx. 0.2±0.3 uM (Biochem. J. 2002, 362, 499-505; Biophysical Journal, 2009, 4887-4895; Cellular Signalling, 2002, 1005-1013)

Another protein identified in this experiment was DECR1 as a putative Akt partner. It has been already reported that DECR1 co-immunoprecipitated with EGFR (Molecular & Cellular Proteomics, 2009, 2595-2612). Protein identified here is G3BP1 as a putative Akt partner. It has been already reported that subsets of EGFR precipitated with PLC-SH2 showed an enhanced association with GTPase-activating protein, PI3-kinase, and SHPTP2/syp)

Protein identified in this experiment was ACLY as a putative Akt partner. It has been previously reported that ACLY involves downstream signaling pathways involving PI3K/AKT and MAPK pathways (Journal of Cellular Physiology, 2011). It has been also previously reported that MYL6B involves downstream EGFR signaling pathways involving PI3K/AKT and MAPK pathways (Molecular & Cellular Proteomics, 2007, 908-922; Molecular Cancer, 2011, 10:79).

In the case of proteins identified by using EGFR antibody, only ERBB4 was found to bind to the EGFR (Molecular Systems Biology, 2005).

Mass spectrometry analysis EGFR co-precipitates allowed the identification of 5 proteins. Among them, 3 proteins have previously been reported through proteomic studies as described above. 2 proteins which may potentially interact with EGFR that are discussed in the instant experiment require further experiments to confirm their functional interactions.

In conclusion, the proteomics exploration of EGFR signaling A431 cells led to the demonstration that the aptamer based Co-AP method can be a useful tool for the identification of physiologically relevant protein-protein interactions study.

### Example 16

### Comparison of Performance of Aptamer and Antibody in Pull-Down Assay

To compare the performance of aptamers and antibodies in pull-down assays, pull-down experiments were performed for both. Methods were the same as described above. Briefly, whole cell lysates from A431 cell lysates corresponding to 100 μg of total cellular proteins that express the EGFR were clarified by centrifugation at 12,000×g for 10 min after brief sonication. The lysates were incubated with either biotinylated 20 pmol EGFR aptamers (#1193-50) or protein A beads coupled anti-EGFR Abs (R&D, DYC1854—part no. 841464) followed by incubation with 1 mM dextran sulfate. The mixed solution was further incubated with 10 μl Streptavidin magnetic beads. After washing the beads, the bound complex was eluted by either boiling SDS loading buffer or 20 mM 20 mM EDTA in 40 mM HEPES containing 0.1M NaCl. The eluate was loaded on a SDS-PAGE (4-15% gradient gel). Protein bands on gel were directly detected by SYPRO ruby stain.

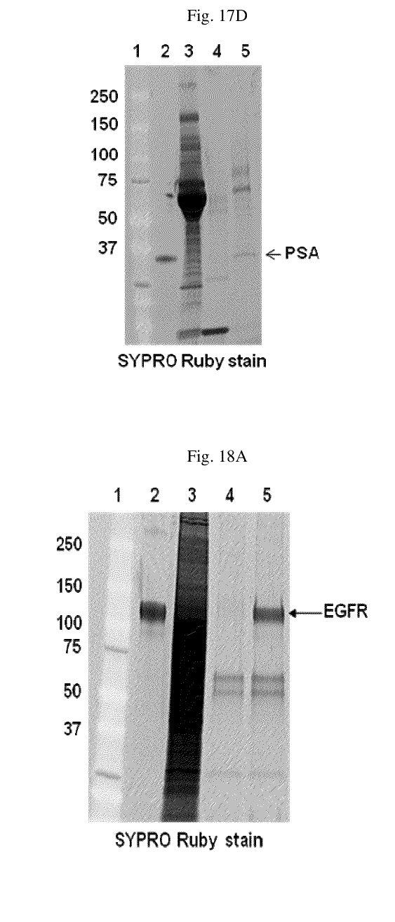

The results are shown in FIG. 14, wherein Lane 1 indicates protein marker; Lane 2 indicates A431 whole cell lysate corresponding to 20 μg of total cellular proteins; Lane 3 indicates blank; Lane 4 indicates sample by boiling SDS loading buffer from the beads bound complex after AP with the biotinylated EGFR-aptamers; Lane 5 indicates eluate by 20 mM EDTA buffer from the beads bound complex; Lane 6 indicates a sample obtained by boiling SDS loading buffer from the beads after elution with 20 mM EDTA buffer; and Lane 7 indicates eluate by boiling SDS loading buffer after AP with the anti-EGFR-antibodies.

As shown in FIG. 14, EGFR aptamers were highly specific to EGFR, while anti-EGFR Abs precipitated extraordinarily small amounts of EGFR. These results indicate that aptamers demonstrate less interference and higher specificity than Abs.

To compare the performance of aptamers and antibodies, pull-down experiments were performed with Rat-1/IR cell overexpressing insulin receptors. Methods were the same as stated above. Pull-down assay was performed with either biotinylated IR-aptamers (#1652-36) or protein A beads coupled anti-IR Abs (R&D, DYC1544, part no. 841872). Insulin receptor is a transmembrane receptor that is activated by insulin. Two alpha subunits and two beta subunits make up the insulin receptor. The beta subunits pass through the cellular membrane and are linked by disulfide bonds.

More specifically, Rat-1/IR cell lysates corresponding to 100 μg of total cellular proteins that overexpress the human IR were incubated with 20 pmol biotinylated IR-aptamers and 1 mM dextran sulfate after 10 μl strepavidin magnetic beads, followed by incubation with 10 μl strepavidin magnetic beads. Elution was performed by 20 mM EDTA in 40 mM HEPES containing 0.1M NaCl.

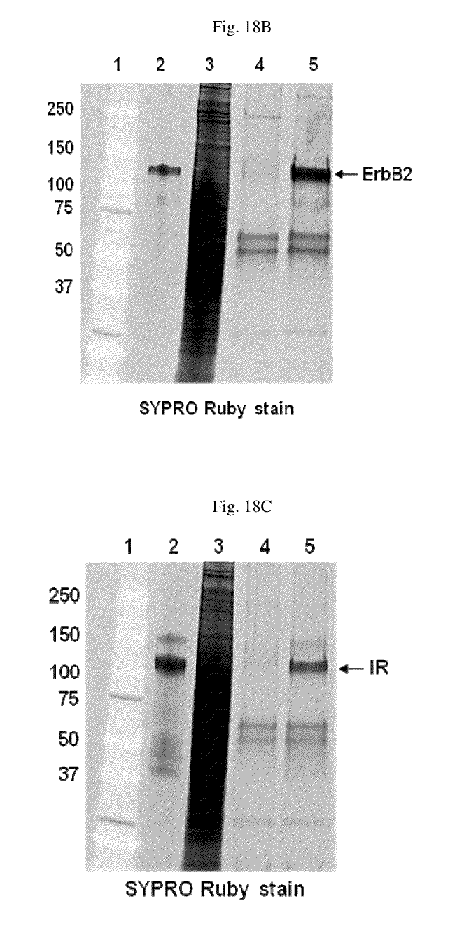

The results are shown in FIG. 15, wherein Lane 1 indicates protein marker; Lane 2 indicates Rat-1/IR whole cell lysate corresponding to 20 μg of total cellular proteins; Lane 3 indicates sample by boiling SDS loading buffer after AP with the biotinylated control aptamers (reverse complement sequence of IR aptamer); Lane 4 indicates sample by boiling SDS loading buffer from the beads after AP with the biotinylated IR-aptamers; Lane 5 indicates an eluation by 20 mM EDTA from the beads bound complex; Lane 6 indicates a sample obtained by boiling SDS loading buffer from the beads after elution with 20 mM EDTA; Lane 7 indicates sample by boiling SDS loading buffer after IP with protein A agrose beads; and Lane 8 indicates sample by boiling SDS loading buffer after IP with the anti-IR antibodies. Molecular weight of IR, heterodimer: about 200 kDa, α-subunit: about 135 kDa, β-subunit: about 95 kDa.

The results showed that aptamers (IR #1652-36) can specifically interact and from a complex with alpha and beta subunits, while anti-insulin receptor Abs did not recognize beta subunits (FIG. 15). Insulin receptor was eluted by 20 mM EDTA with a high yield and specificity in AP assay using aptamers. This result indicates that the performance of aptamers in terms of specificity and selectivity is significantly superior compared to Abs in AP experiments.

To compare the performance of aptamers and antibodies in AP assays, AP and IP experiments were performed with MCF7 cells stably expressing the human ErbB2. Methods were the same as described above. AP and IP assay were performed with either biotinylated ErbB2 receptor aptamers (#1194-34) or protein A beads coupled anti-ErbB2 Abs (R & D, DYC1129E-part no. 841499)

More specifically, MCF7 cell lysates corresponding to 100 μg of total cellular proteins that express the ErbB2 proteins were incubated with 20 pmol biotinylated ErbB2 proteins aptamers and 1 mM dextran sulfate, followed by incubation with 10 μl strepavidin magnetic beads. Elution was by 40 mM HEPES (pH 8.0) buffer containing 20 mM EDTA and 100 mM NaCl.

The results are shown in FIG. 16, wherein Lane 1 indicates protein marker; Lane 2 indicates MCF7 whole cell lysate corresponding to 20 μg of total cellular proteins; Lane 3 indicates sample by boiling SDS loading buffer after AP with biotinylated control aptamers (reverse complement sequence of ErbB2 aptamer); Lane 4 indicates sample by boiling SDS loading buffer from the beads after AP with the biotinylated ErbB2 aptamers; Lane 5 indicates eluate obtained by elution with 20 mM EDTA from the beads bound complex; Lane 6 indicates sample by boiling SDS loading buffer from the beads after elution with EDTA; Lane 7 indicates sample by boiling SDS loading buffer after IP with protein A agarose beads; and Lane 8 indicates sample by boiling SDS loading buffer after IP with the anti-ErbB2 antibodies.

The results showed that aptamers (ErbB2 #1194-34) can specifically interact ErbB2 proteins, while anti-ErbB2 Abs precipitated small amounts of ErbB2 receptor. This result indicates that the performance of aptamers in terms of specificity and selectivity is significantly superior compared to Abs in AP experiments.

To identify aptaprecipitated target proteins, MALDI-MS/MS analysis was performed. All of eluted target proteins were digested with trypsin. Peptided masses were analyzed by MALDI-MS/MS and the peptide mass data were searched using a Mascot search algorithm. As a result, eluted proteins (indicated in FIG. 14, FIG. 15, and FIG. 16) were identified as EGFR, IR, and ErbB2 proteins with high Mowse score and sequence coverage. These results indicated that aptamers can specifically interact and form a complex with their cognate target proteins.

### Example 17

### AP Performance of Biotinylated Aptamer in Serum

Pull-down assay with biotinylated DNA aptamers was performed to validate the specificity and affinity of aptamers towards target proteins in serum. Methods were the same as described above. Briefly, 10% serum samples (FBS, Hyclone) were mixed with each 10 pmol purified recombinant EGFR, ErbB2, IR, and PSA proteins, followed by incubation with the 20 pmol biotinylated aptamers (EGFR #1193-50, ErbB2 #1194-34, IR #1652-36, PSA #1660-07) and 1 mM dextran sulfate. After washing the beads, the bound complex was eluted by nonbiotinylated each 100 pmol aptamer. The eluate was loaded on a SDS-PAGE (4-15% gradient gel). Protein bands on gel were directly detected by SYPRO ruby stain.

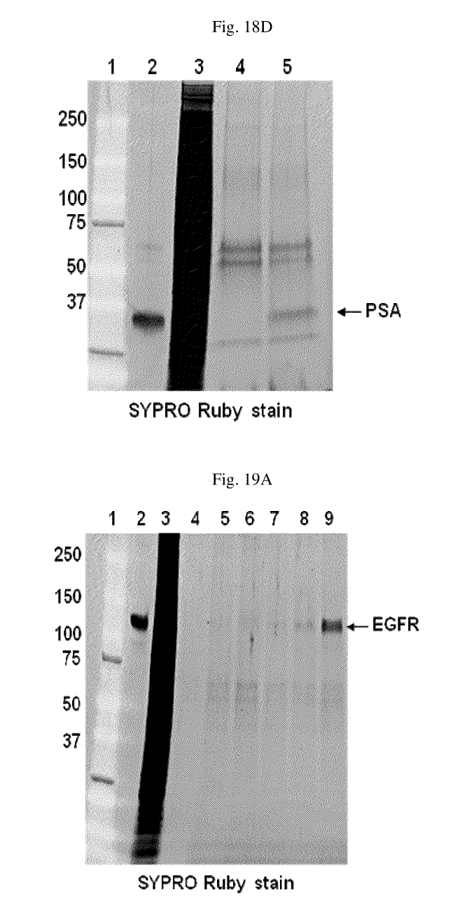

The results are shown in FIGS. 17A, 17B, 17C, and 17D, wherein Lane 1 indicates protein marker; Lane 2 indicates purified recombinant proteins; Lane 3 indicates 10% serum samples mixed with each purified recombinant proteins; Lane 4 indicates sample by boiling SDS loading buffer from the beads after elution with nonbiotinylated aptamer; Lane 5 indicates eluate by nonbiotinylated aptamer from the aptamers-beads bound complex.

All of the each aptamers were highly specific to their cognate target proteins. These results indicated that aptamers demonstrate high specificity in serum. To identify aptaprecipitated target proteins, MALDI-MS/MS analysis was performed. All eluted target proteins were digested with trypsin. Peptide masses were analyzed by MALDI-MS/MS and the peptide mass data were searched using a Mascot search algorithm.

As a result, eluted proteins (indicated in FIGS. 17A, 17B, 17C, and 17D) were identified as EGFR (Mascot score 53, matched peptide 15), IR (Mascot score 200, matched peptide 33), and ERBB2 (Mascot score 104, matched peptide 22), PSA (Mascot score 109, matched peptide 13) respectively (Data not shown). These results indicated that aptamers can specifically interact and form a complex with their cognate target proteins.

### Example 18

### AP Performance of Biotinylated Aptamer in HEK293T Cell

AP assay with biotinylated DNA aptamers was performed to validate the specificity and affinity of aptamers towards target proteins in HEK293T cell lysates. Methods were the same as above. Briefly, HEK293T cell lysates corresponding to 100 μg of total cellular proteins were were mixed with 10 pmol each purified recombinant EGFR, ErbB2, IR, and PSA proteins, followed by incubation with the 20 pmol biotinylated each aptamers (EGFR #1193-50, ErbB2 #1194-34, IR #1652-36, PSA #1660-07) and 1 mM dextran sulfate. After washing the beads, the bound complex was eluted by nonbiotinylated aptamer. The eluate was loaded on a SDS-PAGE (4-15% gradient gel). Protein bands on gel were directly detected by SYPRO ruby stain.

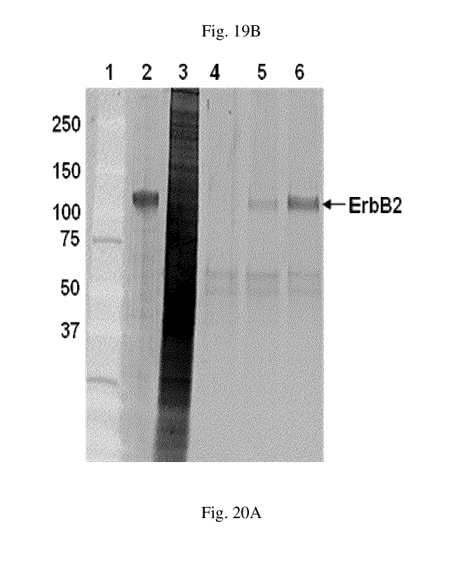

The results are shown in FIGS. 18A-18D, wherein Lane 1 indicates protein marker; Lane 2 indicates purified recombinant proteins; Lane 3 indicates HEK293 whole cell lysate corresponding to 20 μg of total cellular proteins; Lane 4 indicates sample by boiling SDS loading buffer from the beads after AP with the streptavidin beads; and Lane 5 indicates eluate by boiling SDS loading buffer from the beads bound complex after AP with the biotinylated-aptamers/streptavidin beads.

As shown in FIGS. 18A-18D, all aptamers can specifically interact and form a complex with their cognate target proteins. These results indicated that aptamers demonstrate high specificity in HEK293T cell.

### Example 19

### Aptamers-NHS Sepharose Magnetic Beads Conjugate Demonstrate High Specificity in HEK293T Cells

AP assay with conjugated aptamers was performed to validate the specificity and affinity of aptamers towards target receptor proteins in HEK293T cells. In order to prepare direct conjugation between aptamers and beads, aptamers were coupled with NHS-sepharose magnetic beads under proper conditions as described above.

HEK293T cell lysates corresponding to 100 μg of total cellular proteins were mixed with 10 pmol purified recombinant EGFR or ErbB2 proteins, followed by incubation with the conjugated aptamers-sepharose magnetic beads (EGFR #1193-50, ErbB2 #1194-34) and 1 mM dextran sulfate. After washing the beads, the bound complex was eluted by boiling SDS loading buffer. The eluate was loaded on a SDS-PAGE (4-15% gradient gel). Protein bands on gel were directly detected by SYPRO ruby stain.

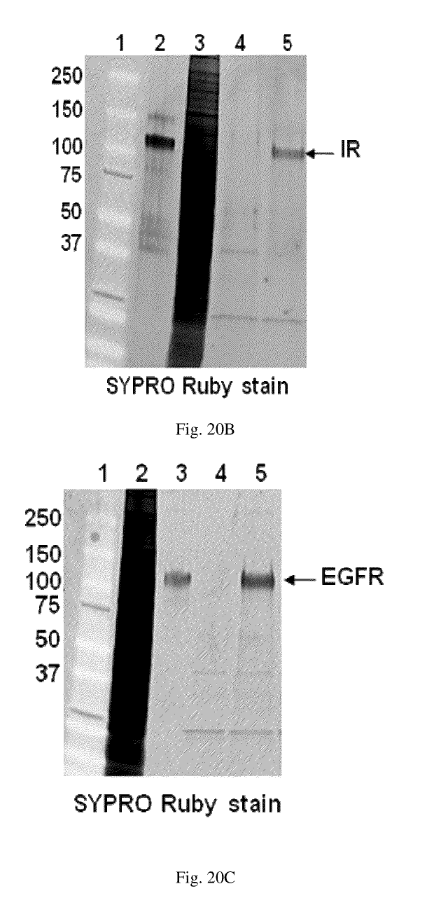

FIG. 19A is SYPRO Ruby stain showing AP of purified recombinant EGFR with the EGFR aptamers-sepharose magnetic beads conjugate. Lane 1 indicates protein marker; Lane 2 indicates purified EGFR proteins; Lane 3 indicates HEK293 cell lysates corresponding to 20 μg of total cellular proteins; Lane 4 indicates Blank; Lane 5 indicates eluate by boiling SDS loading buffer after AP with blank sepharose magnetic beads; Lane 6 indicates eluate by boiling SDS loading buffer after AP with 100 pmol control aptamers (reverse sequence of EGFR aptamer)-sepharose magnetic beads conjugate; Lane 7 indicates eluate by boiling SDS loading buffer after AP with 10 pmol EGFR aptamers-sepharose magnetic beads conjugate; Lane 8 indicates eluate by boiling SDS loading buffer after pull-down with 100 pmol EGFR aptamers-sepharose magnetic beads conjugate; Lane 9 indicates eluate by boiling SDS loading buffer after AP with 1 nmol EGFR aptamers-sepharose magnetic beads conjugate.

FIG. 19B is SYPRO Ruby stain showing pull-down of purified recombinant ErbB2 with the ErbB2 aptamers-sepharose magnetic beads conjugate. Lane 1 indicates protein marker; Lane 2 indicates purified ErbB2 proteins; Lane 3 indicates HEK293 cell lysates corresponding to 20 μg of total cellular proteins; Lane 4 indicates eluate by boiling SDS loading buffer after AP with non-conjugated sepharose magnetic beads; Lane 5 indicates eluate by boiling SDS loading buffer after AP with 100 pmol ErbB2 aptamers-sepharose magnetic beads conjugate; Lane 6 indicates eluate by boiling SDS loading buffer after AP with 1 nmol ErbB2 aptamers-sepharose magnetic beads conjugate.

The amount of the target proteins aptoprecipitated was dependent on the amount of captured aptamers sepharose-magnetic beads conjugates. Both EGFR and ErbB2 aptamers were highly specific to their cognate target proteins. These results indicated that conjugated aptamers could readily bind to their target molecule with high specificity in HEK293T cell lysates.

### Example 20

### Aptamers-Magnetic Agarose Beads Conjugate Demonstrate High Specificity in HEK293T Cells

AP assay with conjugated aptamers was performed to validate the specificity and affinity of aptamers towards target receptor proteins in HEK293T cells. In order to prepare direct conjugation between aptamers and beads, SH-aptamers were coupled with magnetic agarose beads and with maleimide linker under proper conditions as described above.

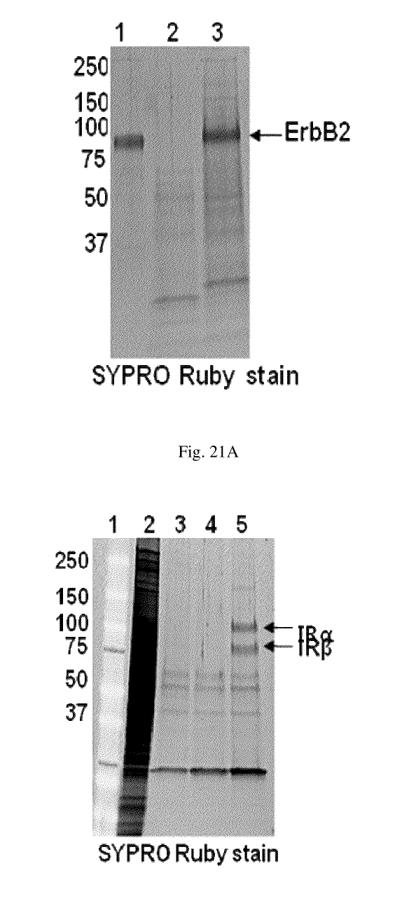

HEK293T cell lysates corresponding to 100 μg of total cellular proteins were mixed with each 10 pmol purified recombinant IR (FIG. 20A), EGFR (FIG. 20B) or ErbB2 (FIG. 20C) proteins after preclering with 200 μg/mL ssDNA, followed by incubation with the conjugated aptamers-magnetic agarose beads (IR #1652-36, EGFR #1193-50, ErbB2 #1194-34) and 1 mM dextran sulfate. After washing the beads, the bound complex was eluted by boiling SDS loading buffer. The eluate was loaded on a SDS-PAGE (4-15% gradient gel). Protein bands on gel were directly detected by SYPRO ruby stain.

FIG. 20A is SYPRO Ruby stain showing AP of purified recombinant IR with the IR aptamers-magnetic agarose beads conjugate. Lane 1 indicates protein marker; Lane 2 indicates purified IR proteins; Lane 3 indicates HEK293 cell lysates corresponding to 20 μg of total cellular proteins; Lane 4 indicates eluate by boiling SDS loading buffer after pull-down with control aptamer (reverse sequence of IR aptamer)-magnetic agarose beads; Lane 5 indicates eluate by boiling SDS loading buffer after AP with 100 pmol IR aptamers-magnetic agarose beads.

FIG. 20B is SYPRO Ruby stain showing AP of purified recombinant EGFR with the EGFR aptamers-magnetic agarose beads conjugate. Lane 1 indicates protein marker; Lane 2 indicates HEK293 cell lysates corresponding to 20 μg of total cellular proteins; Lane 3 indicates purified EGFR proteins; Lane 4 indicates eluate by boiling SDS loading buffer after AP with control aptamer (reverse sequence of EGFR aptamer)-magnetic agarose beads; Lane 5 indicates eluate by boiling SDS loading buffer after pull-down with 100 pmol EGFR aptamers-magnetic agarose beads.

FIG. 20C is SYPRO Ruby stain showing AP of purified recombinant ErbB2 with the ErbB2 aptamers-magnetic agarose beads conjugate. Lane 1, purified ErbB2 proteins; Lane 2, eluate by boiling SDS loading buffer after AP with control aptamer (reverse sequence of ErbB2 aptamer)-magnetic agarose beads; Lane 3, eluate by boiling SDS loading buffer after AP with 100 pmol ErbB2 aptamers-magnetic agarose beads.

All tested IR, EGFR and ErbB2 aptamers were highly specific to their cognate target proteins. These results indicated that conjugated aptamers could readily bind to their target molecule with high specificity in HEK293T cell lysates.

### Example 21

### Aptamers-Magnetic Agarose Beads Conjugate Demonstrate High Specificity in Target Protein Expressing Cells

AP assay with conjugated aptamers was performed to validate the specificity and affinity of aptamers towards target receptor proteins in target protein expressing cells. In order to prepare direct conjugation between aptamers and beads, SH-aptamers were coupled with magnetic agarose beads and with a maleimide linker under proper conditions as described above.

Methods were the same as described above. Briefly, whole cell lysates from Rat-1/IR, A431, and MCF7/ErbB2 cell lines that express the IR, EGFR, and ErbB2 receptors were clarified by centrifugation at 12,000×g for 10 min after brief sonication, followed by preclearing with magnetic agarose beads and 200 μg/mL ssDNA. The precleared lysates were incubated with target-specific aptamers-magnetic agarose beads followed by incubation with dextran sulfate (1.0 mM). After washing the beads, the bound complex was eluted by boiling SDS loading buffer. The eluate was loaded on a SDS-PAGE (4-15% gradient gel). Protein bands on gel were directly detected by SYPRO ruby stain.

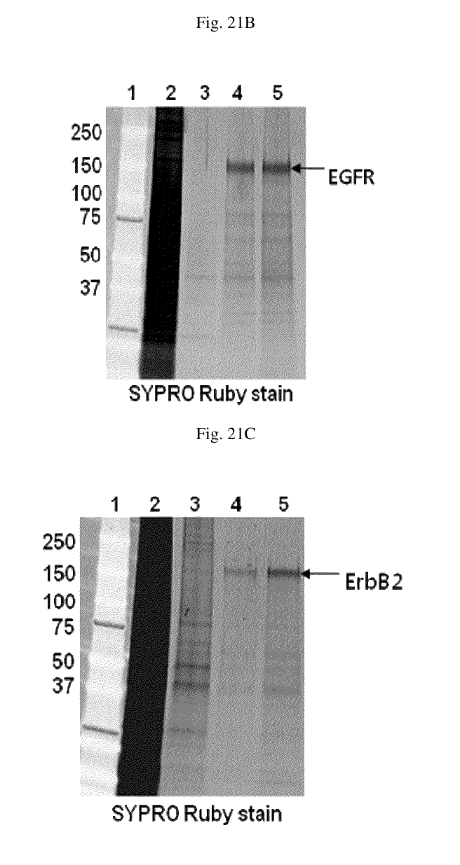

The results are shown in FIG. 21A, wherein Lane 1 indicates protein marker; Lane 2 indicates Rat-1/IR cell lysates corresponding to 20 μg of total cellular proteins; Lane 3 indicates eluate by boiling SDS loading buffer after AP with non-conjugated aptamer-magnetic agarose beads; Lane 4 indicates eluate by boiling SDS loading buffer after AP with control aptamer (reverse sequence of IR aptamer)-magnetic agarose beads; Lane 5 indicates eluate by boiling SDS loading buffer after AP with 100 pmol IR aptamers-magnetic agarose beads.

The results are shown in FIG. 21B, wherein Lane 1 indicates protein marker; Lane 2 indicates A431 cell lysates corresponding to 20 μg of total cellular proteins; Lane 3 indicates eluate by boiling SDS loading buffer after AP with non-conjugated aptamer-magnetic agarose beads; Lane 4 indicates eluate by boiling SDS loading buffer after AP with 50 pmol EGFR aptamers-magnetic agarose beads; Lane 5 indicates eluate by boiling SDS loading buffer after pull-down with 100 pmol EGFR aptamers-magnetic agarose beads.

The results are shown in FIG. 21C, wherein Lane 1 indicates protein marker; Lane 2 indicates MCF7/ErbB2 cell lysates corresponding to 20 μg of total cellular proteins; Lane 3 indicates eluate by boiling SDS loading buffer after AP with non-conjugated aptamer-magnetic agarose beads; Lane 4 indicates eluate by boiling SDS loading buffer after AP with 50 pmol ErbB2 aptamers-magnetic agarose beads; Lane 5 indicates eluate by boiling SDS loading buffer after AP with 100 pmol ErbB2 aptamers-magnetic agarose beads.

IR, EGFR and ErbB2 aptamers were highly specific to their cognate target proteins. These results indicated that conjugated aptamers could readily bind to their target molecules with high specificity.

### Example 22

### Comparison of Performance of Aptamer and Antibody Using Aptamers-Magnetic Agarose Beads Conjugate in Pull Down Assay

### 12.1: Pull-Down Assay Using EGFR Aptamer and EGFR Antibody

To compare the performance of aptamers and antibodies in pull-down assays, pull-down experiments were performed for both. Methods were the same as described above. Briefly, whole cell lysates from A431 cell lines that express the EGFR were clarified by centrifugation at 12,000×g for 10 min after brief sonication, followed by preclearing with magnetic agarose bead and 200 μg/mL ssDNA. The precleared lysates were incubated with either EGFR aptamers-magnetic agarose beads or Dynabead M270 coupled anti-EGFR Abs (Santa cruz-#sc-120, cell signaling-#2232) followed by incubation with dextran sulfate (1.0 mM). After washing the beads, the bound complex was eluted by either boiling SDS loading buffer or 20 mM EDTA. The eluate was loaded on a SDS-PAGE (4-15% gradient gel). Protein bands on gel were directly detected by SYPRO ruby stain. EGFR was eluted by 20 mM EDTA with high yield and specificity.

More specifically, A431 cell lysates corresponding to 1 mg of total cellular proteins that express the EGFR were incubated with either 50 pmol EGFR aptamers or anti-EGFR Abs and 1 mM dextran sulfate after preincubation with 200 μg/mL ssDNA and 10 μl magnetic agarose beads. Elution was by 20 mM EDTA in 40 mM HEPES containing 0.1M NaCl.

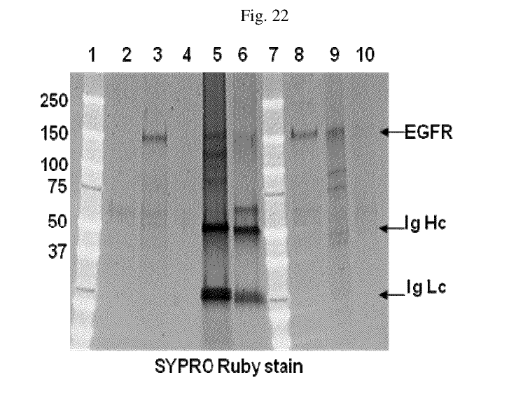

The results are shown in FIG. 22, IP and AP were performed with an antibodies and an aptamer against EGFR, respectively. Proteins bound to resins were analyzed by SDS-PAGE. Lanes 1 and 7 indicate protein marker; Lane 2 indicates eluate by boiling SDS loading buffer after pull down with non-conjugated aptamer-magnetic agarose beads; Lane 3 indicates eluate by boiling SDS loading buffer after AP with 50 pmol EGFR aptamers-magnetic agarose beads; Lane 4 indicates eluate by boiling SDS loading buffer after pull down with non-conjugated-Dynabead M270; Lane 5 indicates eluate by boiling SDS loading buffer after IP with 50 pmol anti-EGFR Ab(Santa cruz, #sc-120)-Dynabeads; Lane 6 indicates eluate by boiling SDS loading buffer after IP with 50 pmol anti-EGFR Ab(cell signaling, #2232)-Dynabeads; Lane 8 indicates eluate by 20 mM EDTA buffer from the beads bound complex after AP with 50 pmol EGFR aptamers-magnetic agarose beads; Lane 9 indicates eluate by elution buffer (supplied by Invitrogen) from the beads bound complex after IP with 50 pmol anti-EGFR Ab(Santa cruz, #sc-120)-Dynabeads; Lane 10 indicates eluate by elution buffer (supplied by Invitrogen) from the beads bound complex after IP with 50 pmol anti-EGFR Ab(cell signaling, #2232)-Dynabeads.

As shown in FIG. 22, it should be noted that many proteins which are not related with EGFR were detected in IP from total proteins bound to resin (lanes 5-6) and the proteins eluted from the resin (lane 9). Moreover, EGFR protein was not clearly detected from IP with antibody. On the other hand, much less non-specific protein-binding was observed in AP total proteins bound to resin (lane 3) and in the proteins eluted from the resin (lane 8) without sacrificing specific binding of EGFR. Collectively, EGFR aptamers were highly specific to EGFR, while anti-EGFR Abs precipitated extraordinarily small amounts of EGFR. These results indicated that aptamers demonstrate less interference and higher specificity than Abs in AP experiment.

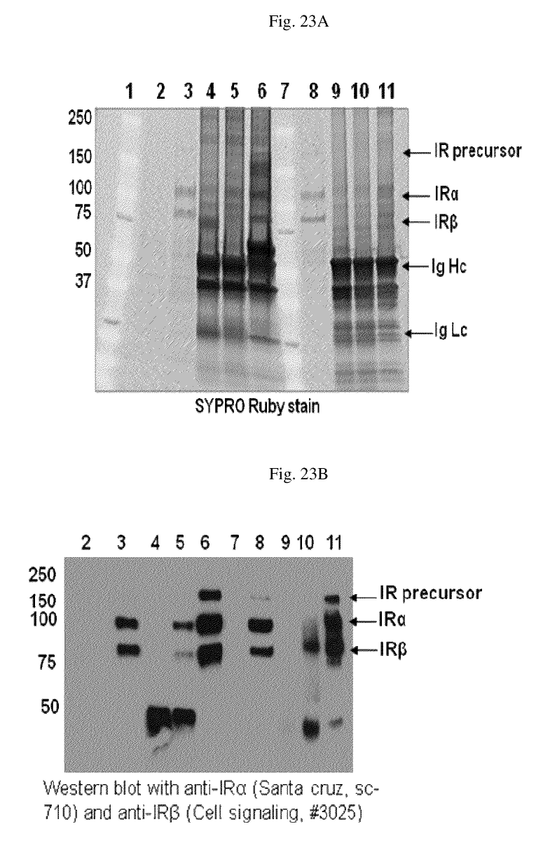

To identify pull-downed target proteins, MALDI-MS/MS analysis was performed. All of eluted target proteins were digested with trypsin. Peptided masses were analyzed by MALDI-MS/MS and the peptide mass data were searched using a Mascot search algorithm. As a result, eluted proteins from aptamer based AP (indicated in FIG. 23A) were identified as EGFR proteins with high Mowse score and sequence coverage, respectively, while eluted proteins from anti-EGFR Abs were identified as EGFR proteins with low Mowse score and sequence coverage. Collectively, peaks matching with EGFR obtained from AP were stronger than those from IP. Moreover, higher scores of MS and MS/MS were observed from proteins obtained by AP. These results indicated that aptamers demonstrate less interference and higher specificity than Abs in AP experiment.

### 22.2: Pull-Down Assay Using IR Aptamer and IR Antibody

To compare the performance of aptamers and antibodies in AP assays, pull-down experiments were performed with another Rat-1/IR cell overexpressing insulin receptors. Methods were the same as described above. Briefly, whole cell lysates from Rat-1/IR cell lines that express the IR were clarified by centrifugation at 12,000×g for 10 min after brief sonication, followed by preclearing with magnetic agarose beads and 200 μg/mL ssDNA. The precleared lysates were incubated with either IR aptamers-magnetic agarose beads or Dynabead M270 coupled anti-IR Abs (Santa cruz anti-IRα (#sc-710), Santa cruz anti-IRβ (#sc-711), Invitrogen anti-Irα (#AHR0221)) followed by incubation with dextran sulfate (1.0 mM). After washing the beads, the bound complex was eluted by either boiling SDS loading buffer or 20 mM EDTA. The eluate was loaded on a SDS-PAGE (4-15% gradient gel). Protein bands on gel were directly detected by SYPRO ruby stain. IR was eluted by 20 mM EDTA with high yield and specificity.

More specifically, Rat-1/IR cell lysates corresponding to 1 mg of total cellular proteins that express the IR were incubated with either 50 pmol IR aptamers or anti-IR Abs and 1 mM dextran sulfate after preincubation with 200 μg/mL ssDNA and 10 μl magnetic agarose beads. Elution was performed by 20 mM EDTA in 40 mM HEPES containing 0.1M NaCl.

The results are shown in FIGS. 23A and 23B, IP and AP were performed with antibodies and an aptamer against insulin receptor (IR), respectively. Proteins bound to resins were analyzed by SDS-PAGE. Total proteins from beads were prepared by addition of SDS sample buffer and boiling (lanes 2-6). Alternatively proteins were eluted with AptSci elusion buffer (lane 8) or with Invitrogen elution buffer (lanes 9-11). Lanes land 7 indicate protein marker; Lane 2 indicates eluate by boiling SDS loading buffer after AP with non-conjugated aptamer-magnetic agarose beads; Lane 3 indicates eluate by boiling SDS loading buffer after pull-down with 50 pmol IR aptamers-magnetic agarose beads; Lane 4 indicates eluate by boiling SDS loading buffer after pull-down with 50 pmol anti-IR Ab(Santa cruz anti-IRα (#sc-710))-Dynabeads; Lane 5 indicates eluate by boiling SDS loading buffer after pull-down with 50 pmol anti-IR Ab(Santa cruz anti-IRβ (#sc-711))-Dynabeads; Lane 6 indicates eluate by boiling SDS loading buffer after AP with 50 pmol anti-IR Ab(Invitrogen anti-Irα (#AHR0221))-Dynabeads; Lane 8 indicates eluate by 20 mM EDTA from the beads bound complex after AP with 50 pmol IR aptamers-magnetic agarose beads; Lane 9 indicates eluate by elution buffer (supplied by Invitrogen) from the beads bound complex after pull-down with 50 pmol anti-IR Ab(Santa cruz anti-IRα (#sc-710))-Dynabeads; Lane 10 indicates eluate by elution buffer (supplied by Invitrogen) from the beads bound complex after pull-down with 50 pmol anti-EGFR Ab(Santa cruz anti-IRβ (#sc-711))-Dynabeads. Lane 11 indicates eluate by elution buffer (supplied by Invitrogen) from the beads bound complex after AP with 50 pmol anti-EGFR Ab(Invitrogen anti-Irα (#AHR0221))-Dynabeads.

As shown in FIG. 23, it should be noted that many proteins which are not related with IR were detected in IP from total proteins bound to resin (lanes 4-6) and the protein eluted from the resin (lanes 9-11). On the other hand, much less non-specific protein-binding was observed in AP total proteins bound to resin (lane 3) and in the proteins eluted from the resin (lane 8) without sacrificing specific binding of IR-alpha and IR-beta. IR-aptamers were highly specific to IR, while anti-IR Abs precipitated extraordinarily small amounts of EGFR. These results indicated that aptamers demonstrate less interference and higher specificity than Abs in AP experiment.

To identify aptoprecipitated target proteins, Western blotting analysis was performed (FIG. 23B). As a result, all IRα, IRβ, and IR precursor were identified in aptamer based AP under both boiled and eluted samples, while any of IR proteins were not detected in antibody (Santa cruz anti-IRα (#sc-710) based pull-down. Santa cruz anti-IRβ (#sc-711) Ab precipitated extraordinarily small amounts of IRα and IRβ under the boiled samples and no IR proteins were identified in eluted samples. However, all IRα, IRβ, and IR precursor were detected by Invitrogen anti-Irα (#AHR0221) under both boiled and eluted samples. These results indicated that aptamers demonstrate less interference and higher specificity than Abs in pull-down experiment.

### 22.3: Pull-Down Assay Using AKT2 Aptamer and AKT2 Antibody

To compare the performance of aptamers and antibodies in pull-down assays, pull-down experiments were performed with MCF7 cell expressing AKT. Akt protein is a serine/threonine protein kinase that plays a key role in multiple cellular processes such as glucose metabolism, cell proliferation, apoptosis, transcription and cell migration.

Methods were the same as described above. Briefly, whole cell lysates from MCF7 cell lines were clarified by centrifugation at 12,000×g for 10 min after brief sonication, followed by preclearing with magnetic agarose beads and 200 μg/mL ssDNA. The precleared lysates were incubated with either AKT2 aptamers-magnetic agarose beads or Dynabead M270 coupled anti-AKT1/AKT2 Abs (Santa cruz, #sc-1619) followed by incubation with dextran sulfate (1.0 mM). After washing the beads, the bound complex was eluted by either boiling SDS loading buffer or 20 mM EDTA. The eluate was loaded on a SDS-PAGE (4-15% gradient gel). Protein bands on gel were directly detected by SYPRO ruby stain. AKT was eluted by 20 mM EDTA with high yield and specificity.

More specifically, MCF7 cell lysates corresponding to 1 mg of total cellular proteins were incubated with either 50 pmol AKT2 aptamers or anti-AKT1/AKT2 Abs and 1 mM dextran sulfate after preincubation with 200 μg/mL ssDNA and 10 μl magnetic agarose beads. Elution was performed by 20 mM EDTA in 40 mM HEPES containing 0.1M NaCl.

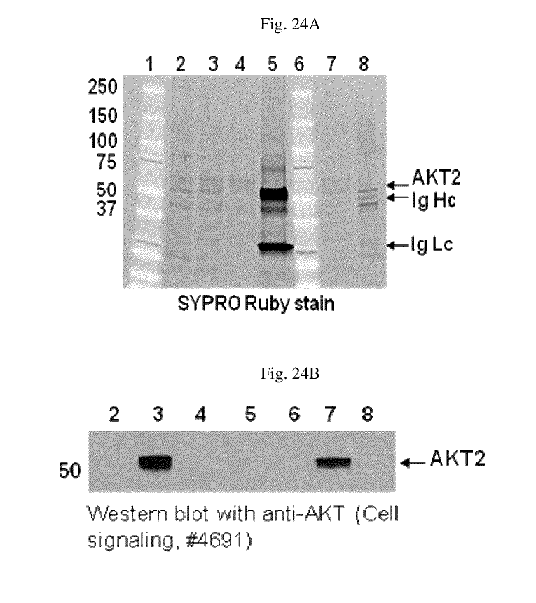

The results are shown in FIG. 24A. IP and AP were performed with an antibody and an aptamer against AKT2, respectively. Proteins bound to resins were analyzed by SDS-PAGE. Lanes land 6 indicate protein marker; Lane 2 indicates eluate by boiling SDS loading buffer after AP with non-conjugated aptamer-magnetic agarose beads; Lane 3, eluate by boiling SDS loading buffer after AP with 50 pmol AKT2 aptamers-magnetic agarose beads; Lane 4, eluate by boiling SDS loading buffer after pull-down with non-conjugated-Dynabead M270; Lane 5, eluate by boiling SDS loading buffer after pull-down with 50 pmol anti-AKT1/AKT2 Ab (Santa cruz, #sc-1619)-Dynabeads; Lane 7, eluate by 20 mM EDTA from the beads bound complex after AP with 50 pmol AKT2 aptamers-magnetic agarose beads; Lane 8 eluate by elution buffer (supplied by Invitrogen) from the beads bound complex after pull-down with 50 pmol anti-AKT1/AKT2 Ab-Dynabeads.

As shown in FIG. 24A (SDS-PAGE) and 24B (Western blot), it has been shown that most of aptamers are useful for protein precipitation, but many antibodies are unable to detect native form of proteins and consequently unable to precipitate target proteins. Only non-specifically bound proteins were observed from AKT2 antibody-conjugated resin. AKT proteins were clearly identified in aptamer based AP under both boiled and eluted samples, while none of AKT proteins was detected in antibody based pull-down under both boiled and eluted samples. These results indicated that aptamers demonstrate less interference and higher specificity than Abs in pull-down experiment.

Because of specificity, sensitive, and accuracy, Co-AP/AP assay is one of the most utilized techniques for routine analysis of target proteins in a variety of applications. For example, the identification of new therapeutically and diagnostically relevant biomolecules can be facilitated. The target molecules can be identified by using aptamer-based Co-AP/AP protocols followed by protease digestion, and liquid chromatography/mass spectrometry analysis. In this regard, aptamer Co-AP/AP assay can be an important tool in variety of proteomic applications. As described above, MS analysis was used to identify target proteins and target protein interacting proteins precipitated by Co-AP/AP assay with their cognate aptamers, followed by elution with either DNase or EDTA, and trypsin digestion. Using the methods described herein, aptamers can be used as capture and detector molecules in Co-AP/AP assays. Such methods can be used in assays where antibody based specific target detection system is utilized such as FACS analysis, ELISA, surface plasmon resonance, and other biomolecule identification assay. Co-AP is a powerful technique that is used regularly by molecular biologists to analyze protein-protein interactions.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of determining a presence of a protein in a fluid sample, said method comprising the steps of:
a) providing a solid substrate bound aptamer, wherein the aptamer is a single-stranded nucleic acid having 20 to 200 nucleotides capable of specifically binding to a target protein;
b) contacting the fluid sample with the solid substrate bound aptamer to form a solid substrate bound aptamer-target protein complex when the target protein is present in the fluid sample; and
c) determining whether the protein is present in the fluid sample by isolating and identifying the target protein from the solid substrate bound aptamer-target protein complex or detecting the formation of the solid substrate bound aptamer-target protein complex, wherein the protein to be determined is the target protein of the aptamer used.

2. The method of claim 1, wherein the protein further comprises an interacting protein of the target protein.

3. The method of claim 1, wherein the fluid sample is at least one selected from serum, spinal fluid, cerebrospinal fluid, joint fluid or one produced by contacting a lysis buffer with at least one selected from mammalian cells, yeasts, virus, or prokaryotic cells.

4. The method of claim 1, wherein the method further comprises a step introduced prior to, after, or simultaneously with step b) of contacting the fluid sample or the solid substrate bound aptamer-target protein complex with at least one selected from an oligonucleotide, or a polymer with charge, to remove undesired proteins.

5. The method of claim 4, wherein the polymer with charge is at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

6. The method of claim 5, wherein the concentration of the polymer with charge in the fluid sample ranges from 0.01 to 10 mM.

7. The method of claim 2, wherein the method further comprises a step introduced prior to, after, or simultaneously with step b) of contacting the fluid sample or the solid substrate bound aptamer-target protein complex with at least one selected from an oligonucleotide, or a polymer with charge, to remove undesired proteins.

8. The method of claim 7, wherein the polymer with charge is at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

9. The method of claim 8, wherein the concentration of the polymer with charge in the fluid sample ranges from 0 to 0.1 mM.

10. A detection kit for determining a presence of a protein in a fluid sample, said kit comprises:
a solid substrate;
an aptamer bound to said solid substrate, wherein the aptamer is a single-stranded nucleic acid having 20 to 200 nucleotides capable of specifically binding to a target protein; and
a detection means for identifying the formation of a solid substrate bound aptamer-target protein complex,
wherein the protein to be detected by the detection means is the target protein of the aptamer used.

11. The detection kit according to claim 10, wherein the protein further comprises an interacting protein of the target protein.

12. The detection kit according to claim 10, wherein the kit further comprises a polymer with charge, an oligonuceleotide, or a combination thereof.

13. The detection kit according to claim 12, wherein the polymer with charge is at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

14. The detection kit according to claim 13, wherein the concentration of the polymer with charge in the fluid sample ranges from 0.01 to 10 mM.

15. The detection kit according to claim 11, wherein the kit further comprises a polymer with charge, an oligonuceleotide, or a combination thereof.

16. The detection kit according to claim 15, wherein the polymer with charge is at least one selected from dextran sulfate, polyanionic cellulose polymer, hyaluronic acid, polyanionic heparin, polysulfonate polymer, polyanionic dendrimer, carboxymethyl-dextran, heparin, aurintricarboxylic acid, or suramin.

17. The detection kit according to claim 16, wherein the concentration of the polymer with charge in the fluid sample ranges from 0 to 0.1 mM.

18. The detection kit according to claim 10, wherein the aptamer is one or more selected from the group consisting of SEQ ID NOs 6 to 10.

19. An oligonucleotide molecule having the nucleotide sequence selected from the group consisting of SEQ ID NOs 6 to 10.
